SEQUENCE LISTING (29 sequences)

SEQ ID NO: 1 - DNA, 24 bp, Artificial Sequence
  Synthetic Polynucleotide
caccgtcctgctgatctccttcgc

SEQ ID NO: 2 - DNA, 24 bp, Artificial Sequence
  Synthetic Polynucleotide
aaacgcgaaggagatcagcaggac

SEQ ID NO: 3 - DNA, 24 bp, Artificial Sequence
  Synthetic Polynucleotide
caccgaaacagacatggccggcga

SEQ ID NO: 4 - DNA, 24 bp, Artificial Sequence
  Synthetic Polynucleotide
aaactcgccggccatgtctgtttc

SEQ ID NO: 5 - DNA, 20 bp, Artificial Sequence
  Synthetic Polynucleotide
ttcggaactgaggccatgat

SEQ ID NO: 6 - DNA, 20 bp, Artificial Sequence
  Synthetic Polynucleotide
tttcgctctggtccgtcttg

SEQ ID NO: 7 - DNA, 20 bp, Artificial Sequence
  Synthetic Polynucleotide
cccctgtaagcgatactcca

SEQ ID NO: 8 - DNA, 20 bp, Artificial Sequence
  Synthetic Polynucleotide
tgggagccattcattttctc

SEQ ID NO: 9 - DNA, 22 bp, Artificial Sequence
  Synthetic Polynucleotide
tcggagatggttccagttatca

SEQ ID NO: 10 - DNA, 21 bp, Artificial Sequence
  Synthetic Polynucleotide
attctggcgttcttcatggtt

SEQ ID NO: 11 - DNA, 19 bp, Artificial Sequence
  Synthetic Polynucleotide
gcggtgcgctttgaacaag

SEQ ID NO: 12 - DNA, 21 bp, Artificial Sequence
  Synthetic Polynucleotide
acactttgtacgggtctgaga

SEQ ID NO: 13 - DNA, 21 bp, Artificial Sequence
  Synthetic Polynucleotide
caacaagatgaagagcaccaa

SEQ ID NO: 14 - DNA, 21 bp, Artificial Sequence
  Synthetic Polynucleotide
gcccacgaatagttgtcattt

SEQ ID NO: 15 - DNA, 21 bp, Artificial Sequence
  Synthetic Polynucleotide
gcttgcaacttgaatctgata

SEQ ID NO: 16 - DNA, 21 bp, Artificial Sequence
  Synthetic Polynucleotide
gcatttggaaacgggaataaa

SEQ ID NO: 17 - DNA, 21 bp, Artificial Sequence
  Synthetic Polynucleotide
ctacgagaaggagaacgaaat

SEQ ID NO: 18 - DNA, 21 bp, Artificial Sequence
  Synthetic Polynucleotide
ccgcttccacatgagctaaag

SEQ ID NO: 19 - DNA, 21 bp, Artificial Sequence
  Synthetic Polynucleotide
gcctgaaatcccttacagcta

SEQ ID NO: 20 - DNA, 21 bp, Artificial Sequence
  Synthetic Polynucleotide
gtaacctcctccgccacatta

SEQ ID NO: 21 - DNA, 21 bp, Artificial Sequence
  Synthetic Polynucleotide
gacagtctaagagtaagtaaa

SEQ ID NO: 22 - DNA, 20 bp, Artificial Sequence
  Synthetic Polynucleotide
agttgcaggttgacctacgg

SEQ ID NO: 23 - DNA, 20 bp, Artificial Sequence
  Synthetic Polynucleotide
agctttcacccgttgagctt

SEQ ID NO: 24 - DNA, 20 bp, Artificial Sequence
  Synthetic Polynucleotide
actctcagtttcaccgctcg

SEQ ID NO: 25 - DNA, 20 bp, Artificial Sequence
  Synthetic Polynucleotide
ctccgggtcctctctggtat

SEQ ID NO: 26 - DNA, 20 bp, Artificial Sequence
  Synthetic Polynucleotide
cgtggctatgtttgcttggg

SEQ ID NO: 27 - DNA, 20 bp, Artificial Sequence
  Synthetic Polynucleotide
agcaggcctcttggttgttt

SEQ ID NO: 28 - DNA, 20 bp, Artificial Sequence
  Synthetic Polynucleotide
gcagtgctgacactggatct

SEQ ID NO: 29 - DNA, 20 bp, Artificial Sequence
  Synthetic Polynucleotide
gcctgccatgcgtaatcaag

(19) United States
(12) Patent Application Publication
(10) Pub. No.: US 2017/0292959 A1
(43) Pub. Date: Oct. 12, 2017

(54) PLASMIDS COMPRISING INTERNAL RIBOSOMAL ENTRY SITES AND USES THEREOF

(71) Applicant: Dana-Farber Cancer Institute, Inc., Boston, MA (US)

(72) Inventors: William G. Kaelin, Boston, MA (US); Gang Lu, San Diego, CA (US); Richard Middleton, Acton, MA (US); Kwok-kin Wong, Arlington, MA (US); James e. Bradner, Weston, MA (US)

(73) Assignee: Dana-Farber Cancer Institute, Inc., Boston, MA (US)

(21) Appl. No.: 15/518,039

(22) PCT Filed: Oct. 9, 2015
(86) PCT No.: PCT/US15/54893

§ 371 Date: Apr. 10, 2017

Related U.S. Application Data
(60) Provisional application No. 62/062,245, filed on Oct. 10, 2014.
(60) Provisional application No. 62/154,858, filed on Apr. 30, 2015.

(51) Int. Cl. G01N 33/68; C12N 15/113; C12N 15/10; C12N 15/64

(52) U.S. Cl. CPC G01N 33/6845; C12N 15/64; C12N 15/1137; C12N 15/1082; C12N 2310/20; C12N 2330/51; G01N 2500/10; C12N 2310/14

(57) ABSTRACT

The present application relates, in some aspects, to the development of a plasmid that can be used to efficiently monitor the stabilities of thousands of proteins after specific perturbations. The plasmid allows for the co-expression of two reporter proteins, each of which is placed under the control of an IRES. Other aspects of the invention relate to a plasmid library, screening methods to identify proteins whose levels are modulated by a compound of interest, and methods for monitoring treatment of a subject with an IMiD compound.

### RELATED APPLICATIONS

This application claims the benefit under 35 U.S.C. §119(e) of U.S. provisional application No. 62/154,858, filed Apr. 30, 2015, and U.S. provisional application No. 62/062,245, filed Oct. 10, 2014, the entire contents of each application are incorporated herein by reference.

### FEDERALLY SPONSORED RESEARCH

This invention was made with government support under 2R01CA068490-19, and 2R01CA076120-13 awarded by National Institute of Health. The government has certain rights in the invention.

### BACKGROUND OF THE INVENTION

Reporter assays have been used routinely in the pharmaceutical and biotechnology industries to identify lead compounds that affect protein function. In the last decade, the chemist's ability to synthesize large numbers of chemical compounds in a short amount of time through techniques such as combinatorial chemistry has greatly increased, and often, thousands to millions of compounds need to be screened to identify those having a desired effect on a protein of interest.

Typically, reporter assays measure the activities of one reporter protein in a sample, but may combine multiple reporters. One strategy for co-expression of multiple reporters involves the design of bicistronic constructs, in which two genes separated by an internal ribosome entry site (IRES) sequence are expressed as a single transcriptional cassette (or bicistronic transcript) under the control of a common upstream promoter (Yen et al., Science. 2008 Nov. 7; 322(5903):918-23). The intervening IRES sequence functions as a ribosome-binding site for efficient cap-independent internal initiation of translation. Such a design enables transcription of both genes with IRES-directed cap-independent translation. This system allows for co-expression of both a control reporter, not expected to change upon experimental treatment, along with a test reporter that is normalized to the control in each test sample. However, many perturbations in the cell can differentially affect cap-dependent translation compared to cap-independent translation. Moreover, some IRESes have been shown to display variable expression of the downstream gene (Wong et al. Gene Ther. 2002 Mar.; 9(5):337-44). This leads to high false positives and unreliable reporter assays. Thus, there is a need for an efficient high-throughput approach for analysis of protein stability where nonspecific alterations in reporter activity are used to control for the inherent variability in cell based protein stability assays. This allows for reducing the error in the data required to effectively and efficiently run an HTS assay.

### SUMMARY OF THE INVENTION

The present disclosure relates, in some aspects, to the development of plasmid that can be used to efficiently monitor the stabilities of thousands of proteins after specific perturbations.

According to some aspects, the present disclosure provides a DNA plasmid. The plasmid comprises in operable linkage a promoter; a first internal ribosomal entry site (IRES); a nucleotide sequence encoding a first reporter protein; a second IRES; and a nucleotide sequence encoding a second reporter protein, wherein an open reading frame (ORF) is fused to the nucleotide sequence encoding a first reporter protein or to the nucleotide sequence encoding a second reporter protein.

In some embodiments, the first and second reporter proteins have distinguishable detectable reporter signals. In some embodiments, the first and second reporter proteins are enzyme proteins having distinguishable signals generated from their products. In some embodiments, the first and second reporter proteins are bioluminescent proteins having distinguishable bioluminescence signals. In some embodiments, the first and second reporter proteins are fluorescent proteins having distinguishable fluorescence signals. In some embodiments, the first and second reporter proteins are selected from the group consisting of renilla luciferase (Rluc) and firefly luciferase (FLuc). In some embodiments, the first and second reporter proteins are selected from the group consisting of green fluorescence protein and red fluorescence protein. In some embodiments, the promoter is a eukaryotic promoter or a synthetic promoter. In some embodiments, the promoter comprises cytomegalovirus (CMV) promoter. In some embodiments, the open reading frame is derived from an ORFeome of an organism. In some embodiments, the open reading frame encodes an oncoprotein. In some embodiments, the oncoprotein is selected from the group consisting of MYC, Ikaros family zinc finger protein 1 (IKZF1), Ikaros family zinc finger protein 3 (IKZF3), Interferon regulatory factor 4 (IRF4), mutant p53, N-Ras, c-Fos, and c-Jun.

Some aspects of the disclosure relates to an isolated transformed host cell comprising the plasmid described herein. In some embodiments, the host cell is a bacterial cell, a yeast cell, a plant cell, an insect cell, or a mammalian cell.

According to some aspects, the present disclosure provide a DNA plasmid library comprising a plurality of plasmids, wherein each said plasmid comprises in operable linkage a promoter; a first internal ribosomal entry site (IRES);a nucleotide sequence encoding a first reporter protein; a second IRES; and a nucleotide sequence encoding a second reporter protein, wherein an open reading frame is fused to the nucleotide sequence encoding a first reporter protein or to the nucleotide sequence encoding a second reporter protein.

In some embodiments, the open reading frame of each plasmid is different. In some embodiments, the open reading frame of each plasmid is derived from an ORFeome of an organism. In some embodiments, the organism is a mammal. In some embodiments, the mammal is human. In some embodiments, the first and second reporter proteins have distinguishable detectable reporter signals. In some embodiments, the first and second reporter proteins are bioluminescent proteins having distinguishable bioluminescence signals. In some embodiments, the first and second reporter proteins are fluorescent proteins having distinguishable fluorescence signals. In some embodiments, the first and second reporter proteins are selected from the group consisting of renilla luciferase (Rluc) and firefly luciferase (FLuc). In some embodiments, the first and second reporter proteins are selected from the group consisting of green fluorescence protein and red fluorescence protein. In some embodiments, the promoter is a eukaryotic promoter or a synthetic promoter. In some embodiments, the promoter comprises cytomegalovirus (CMV) promoter.

According to some aspects, the present disclosure provides a method for identifying proteins whose levels are modulated by a compound of interest, the method comprising: contacting host cells transformed with the plasmid library described herein with a compound of interest; determining ratios of fused reporter protein signal to unfused reporter protein signal in presence and absence of the compound; identifying open reading frames that have increased levels when the ratio of fused reporter protein signal to unfused reporter protein signal in the presence of the compound is increased as compared to the ratio of fused reporter protein signal to unfused reporter protein signal in the absence of the compound and identifying open reading frames that have decreased levels when the ratio of fused reporter protein signal to unfused reporter protein signal in the presence of the compound is decreased as compared to the ratio of fused reporter protein signal to unfused reporter protein signal in the absence of the compound.

In some embodiments, contacting host cells transformed with the plasmid library described herein with a compound of interest comprises growing the transformed host cells in the presence of the compound for an appropriate time. In some embodiments, the compound of interest is an IMiDs®.

According to some aspects, the present disclosure provides a method for monitoring treatment of a subject with an IMiD compound, the method comprising: determining in a sample of a subject treated with an IMiD compound a level of IKZF1 and/or IKZF3; and identifying the subject as responding to the treatment when the level of IKZF1 and/or IKZF3 is decreased as compared to a reference level.

In some embodiments, the reference level is the level of IKZF1 and/or IKZF3 in a control subject that has not been treated with the IMiD compound.

Each of the embodiments and aspects of the invention can be practiced independently or combined. Also, the phraseology and terminology used herein is for the purpose of description and should not be regarded as limiting. The use of “including”, “comprising”, or “having”, “containing”, “involving”, and variations thereof herein, is meant to encompass the items listed thereafter and equivalents thereof as well as additional items.

These and other aspects of the inventions, as well as various advantages and utilities will be apparent with reference to the Detailed Description. Each aspect of the invention can encompass various embodiments as will be understood.

All documents identified in this application are incorporated in their entirety herein by reference.

### BRIEF DESCRIPTION OF THE DRAWINGS

The accompanying drawings are not intended to be drawn to scale. In the drawings, each identical or nearly identical component that is illustrated in various figures is represented by a like numeral. For purposes of clarity, not every component may be labeled in every drawing. In the drawings:

FIGS. 1A-1E show the down-regulation of IKZF1 and IKZF3 by lenalidomide. (FIG. 1A) Vector schematic. (FIG. 1B) Distribution of fold change in Fluc/Rluc ratios after lenalidomide (LEN) (2 μM) treatment. (FIGS. 1C and 1D) Fluc/Rluc ratios (top panels) and immunoblots (bottom panels) of 293FT cells transfected to produce the indicated IKZF proteins fused to Fluc (top panels) or HA tag (bottom panels). Where indicated, cells were treated with lenalidomide (2 μM), MLN4924 (1 μM), or MG132 (10 μM) for 12 hours. Fluc/Rluc ratios were normalized to corresponding dimethyl sulfoxide (DMSO)-treated cells. Data are presented as mean±SD (n=4). (FIG. 1E) Immunoblot analysis of MM1S and L363 cells treated with LEN (2 μM) and MLN4924 (1 μM), as indicated, for 12 hours.

FIGS. 2A-2D depicts that the down-regulation of IKZF1 and IKZF3 by lenalidomide requires cereblon. (FIG. 2A) Immunoblot analysis of 293FT cells stably infected with lentiviral vectors expressing the indicated IKZF-HA proteins and a doxycycline-inducible CRBN shRNA. Where indicated, LEN (2 μM) and doxcycyline (Dox) (1 μg/ml) were added for 12 and 60 hours, respectively. (FIG. 2B) Fluc/Rluc ratios (top panels) and immunoblots (bottom panels) of CRBN+/+ and CRBN−/− 293FT cells transfected to produce IKZF1 fused to Fluc (top panel) or HA tag (bottom panels). Where indicated cells were treated with LEN (2 μM) for 12 hours. Fluc/Rluc ratios were normalized to corresponding DMSO-treated cells. Data are presented as mean±SD (n=4). (FIGS. 2C and 2D) Immunoblot analysis of CRBN+/+ and CRBN−/− MM1S myeloma cells. Where indicated, cells were treated with LEN (2 μM) for 24 hours (FIG. 2C) or 1 hour before the addition of cyclohexamide (CHX) (100 μg/ml) for the indicated periods (FIG. 2D).

FIGS. 3A-3F shows that lenalidomide promotes ubiquitylation of IKZF1 and IKZF3 by cereblon. (FIGS. 3A and 3B) FLAG-IKZF was immunoprecipitated from CRBN−/− 293FT cells stably infected to produce the indicated IKZF proteins and used to capture cereblon from CRBN+/+ 293FT cells (FIG. 3A) or CRBN−/− 293FT cells transfected to produce the indicated CRBN variants (FIG. 3B). Cells were treated with LEN (2 μM) for 12 hours before lysis, as indicated. Bound proteins were detected by immunoblot analysis. (FIG. 3C) Immunoblot analysis of proteins captured with nickel Sepharose from 293FT cells transfected to produce the indicated FLAG-, His-, and V5-tagged proteins. The cells were treated with MG132 (10 μM) and, where indicated, with LEN (2 μM) for 12 hours. (FIG. 3D) CRBN−/− 293FT cells were transfected to produce IKZF1-HA and the indicated Myc-cereblon variants and lysed. The extracts were mixed, treated with LEN (2 μM) or DMSO, and immunoprecipitated with antibodies against HA (anti-HA) or anti-Myc. The immunoprecipitates were incubated with recombinant E1, E2, and ubiquitin (Ub) and subjected to immunoblot analysis.

FIG. 4 depicts antimyeloma activity of lenalidomide linked to loss of IKZF1 and IKZF3. (FIGS. 4A and 4B) Immunoblot analysis (FIG. 4A) and proliferation (FIG. 4B) of myeloma cell lines treated with LEN (2 μM) for the indicated periods. In (FIG. 4B), data are presented as mean±SD (n=4). (FIG. 4C) Change in % red fluorescent protein (RFP) positivity over time in MM1S cells infected with viruses encoding RFP and the indicated shRNAs. The day 2% RFP for each virus was normalized to 1, and subsequent values were expressed relative to cells infected with a virus encoding RFP and a control (CNTL) shRNA. (FIG. 4D) Immunoblot analysis of MM1S cells transiently infected with lentiviruses expressing the indicated shRNAs for 72 hours. (FIG. 4E) MM1S cells were infected with lentiviral vectors encoding GFP and the indicated FLAG-tagged proteins. Shown for each protein is the percentage of GFP positivity for cells treated with LEN (2 μM) for the indicated duration compared to DMSO. (FIG. 4F) Immunoblot analysis of MM1S cells infected as in (FIG. 4E) and treated with DMSO or LEN (2 μM) for 24 hours.

FIGS. 5A-5B show that firefly/renilla luciferase ratios are stable over a range of reporter plasmid concentrations. 293FT cells were transiently transfected with the indicated amounts of plasmids encoding firefly luciferase (Fluc) alone (FIG. 5A) or a HIF1αa-Fluc fusion protein (FIG. 5B). Empty pBluescript-KS was added to bring the total plasmid DNA to 800 ng. Dual luciferase assays were performed 48 hours later and Fluc/Rluc was calculated.

FIGS. 6A-6B depict the pharmacological stabilization of firefly luciferase fusion protein. 293FT cells were transiently transfected with plasmids encoding unfused Fluc (pIRIF) or the indicated ORF-firefly luciferase (Fluc) fusions and then treated for 24 hours with DMOG (1 μM), MG132 (10 μM), or vehicle (DMSO). HIF1α-dPA is a HIF1α variant lacking both prolyl hydroxylation sites (33). Shown for each plasmid are Fluc/Rluc values (FIG. 6A) and Fold change in Fluc/Rluc compared to DMSO treatment (FIG. 6B). Data are mean±SD, n=4.

FIGS. 7A-7B depict the downregulation of IKZF1 and IKZF3 by pomalidomide. Firefly luciferase (Fluc)/renilla luciferase (Rluc) ratios (FIG. 7A) and immunoblots (FIG. 7B) of 293 FT cells transfected to produce the indicated IKZF proteins fused to firefly luciferase (top panel) or hemagglutinin (HA) epitope tag (bottom panels). Where indicated cells were treated with pomalidomide (POM) (0.2 μM) for 12 hours. Fluc/Rluc ratios were normalized to corresponding DMSO treated cells. Data are mean±SD, n=4.

FIG. 8 depicts that the mRNA Level of IKZF1 and IKZF3 is not significantly affected by lenalidomide. Real-time qPCR analysis of MM1S and L363 cells treated with LEN (2 μM) and MLN4924 (1 μM) as indicated for 12 hours. Data are mean±SD, n=3.

FIG. 9 shows monitoring changes in HIF2α stability in response to pVHL. FIG. 9A is a schematic of Bicistronic Reporter Expressing Firefly Luciferase and HIF2α Fused to NanoLuc. NanoLuc contained an HA epitope tag and Firefly Luciferase was partially destabilized by the inclusion of 2 C-terminal degron (PEST) sequences so that the half-life of the internal control reporter (firefly) would be more comparable to Nluc fusions, such Nluc fused to HIF2α, that are inherently unstable. This approach is necessary for certain unstable proteins (e.g. HIF2α) that produce very low Fluc signals as Fluc fusions. FIG. 9B shows NanoLuc to Firefly luciferase values for clonal 786-O VHL−/− renal carcinoma cells containing reporter depicted in (FIG. 9A) that were subsequently infected with doxycycline (DOX)-inducible retroviral expression vectors encoding wild-type pVHL, a tumor-derived pVHL mutant (Y98N), or with the empty virus. Cells were treated with DOX overnight where indicated. FIG. 9C shows Western blot of cells used in FIG. 17B. Note that pVHL Y98N is not completely inactive. FIG. 9D shows activity of 786-O renal carcinoma cells stably expressing HIF2α-Nluc/Firefly Luciferase and treated with cycloheximide (10 μg/ml). HIF2α-Nluc and Luc2CP decay with similar half-lives after treating cells with the protein translation inhibitor cycloheximide. Otherwise the ratio of Nluc and Fluc might change in response to non-specific inhibitors of transcription, translation, or protein degradation.

In FIG. 10 MDA-MB-231 cells stably transfected with a plasmid expressing an IRES-ER-FLuc-IRES-RLuc reporter under the control of a UBC promoter were plated in opaque 96 well plates in DMEM+10% FBS at a concentration of 20,000 cells per well. The following day the cells were treated with the indicated drugs in DMEM+10% FBS for 6 hours at 37 degrees, 10% CO2. The firefly luciferase and renilla signals were quantified using the Dual-Glo Luciferase Assay System (Promega) and a 96 well plate reader (Berthold). Three replicates were plotted per drug concentration. Error bars refer to SD. Note ER ligands affect FLuc without affecting RLuc, in contrast to cycloheximide (CHX).

### DETAILED DESCRIPTION OF THE INVENTION

The present application relates, in some aspects, to the development of a plasmid that can be used to efficiently monitor the stabilities of thousands of proteins after specific perturbations. The plasmid allows for the co-expression of two reporter proteins, each of which is placed under the control of an IRES. In this way both reporters are transcribed together (i.e. are encoded by the same mRNA) and both are translated using an IRES. This minimizes the problem of spurious changes in the ratio of the two reporters caused by perturbations (e.g. compounds) that differentially effect IRES-dependent versus IRES-independent translation, and thus minimizes false positives. Other aspects of the invention relate to a plasmid library, screening methods to identify proteins whose levels are modulated by a compound of interest, and methods for monitoring treatment of a subject with an IMiD compound.

According to one aspect of the invention, a DNA plasmid is provided. The plasmid comprises in operable linkage (a) a promoter, (b) a first internal ribosomal entry site (IRES); (c) a nucleotide sequence encoding a first reporter protein; (d) a second IRES; and (e) a nucleotide sequence encoding a second reporter protein, wherein an open reading frame is fused to the nucleotide sequence encoding a first reporter protein or to the nucleotide sequence encoding a second reporter protein.

As used herein, “operable linkage” refers to a functional linkage between two nucleic acid sequences, such as a transcription control element (e.g., a promoter) and the linked transcribed sequence. Thus, a promoter is in operable linkage with a gene if it can mediate transcription of the gene.

As used herein a “promoter” usually contains specific DNA sequences (responsive elements) that provide binding sites for RNA polymerase and transcriptional factors for transcription to take place. In some embodiments, the promoter is a eukaryotic promoter or a synthetic promoter. Examples of promoters include, but are not limited to, the TATA box, the SV40 late promoter from simian virus 40, cytomegalovirus (CMV) promoter, ubiquitin C promoter (UbC promoter) and the T7 promoter. These and other promoter sequences are well known in the art. In one example of the invention, the promoter is a CMV promoter. In one example of the invention, the promoter is a UbC promoter.

As used herein, an “internal ribosomal entry site” or “IRES” is a cis acting nucleic acid element that mediates the internal entry of ribosomes on an RNA molecule and thereby regulates translation in eukaryotic systems. In the methods and compositions of the present invention, a first and a second IRES elements are contained in the plasmid. The first and second IRES elements permit the independent translation of a nucleotide sequence encoding a reporter protein and an open reading frame fused to a nucleotide sequence encoding another reporter protein from a single messenger RNA. In some embodiments, the first and second IRESs are the same (i.e., they have identical sequences). In some embodiments, the first and second IRESs are not the same (i.e., they do not have identical sequences).

Many IRES elements have been identified in both viral and eukaryotic genomes. In addition, synthetic IRES elements have also been developed. For example, IRES elements have been found in a variety of viruses including members of the genus Enterovirus (e.g. human poliovirus 1 (Ishii et al. (1998) J Virol. 72:2398-405 and Shiroki et al. (1997) J. Virol. 77:1-8), human Coxsackievirus B); Rhinovirus (e.g., human rhinovirus); Hepatovirus (Hepatitis A virus); Cardiovirus (Encephalomyocarditis virus ECMV (nucleotides 2137-2752 of GenBank Accession No. AB041927 and Kim et al. (1992) Mol Cell Biology 72:3636-43) and Etheirler's encephalomyelitis virus); Aphtovirus (Foot-and mouth disease virus (nucleotides 600-1058 of GenBank Accession No. AF308157; Belsham et al. (1990) EMBO 77:1105-10; Poyry et al. (2001) RNA 7:647-60; and Stoneley et al. (2000) Nucleic Acid Research 25:687-94), equine rhinitis A virus, Ewuine rhinitis B); Pestivirus (e.g., Bovine viral diarrhea virus (Poole et al. (1995) Virology 206:150-154) and Classical swine fever virus (Rijnbrand et al. (1997) J. Virol 77:451-7); Hepacivirus (e.g., Hepatitis C virus (Tsukiyama-Kohara et al. (1992) J. Virol. 66:1476-1483, Lemon et al. (1997) Semin. Virol. 5:274-288, and nucleotide 1201-1812 of GenBank Accession No. AJ242654.) and GB virus B). Each of these references is herein incorporated by reference.

IRES elements have also been found in viruses from the family Retroviridae, including members of the Lentivirus family (e.g., Simian immunodeficiency virus (Ohlmann et al. (2000) Journal of Biological Chemistry 275:11899-906) and human immunodeficiency virus 1 (Buck et s/. (2001) J Virol. 75:181-91); the BLV-HTLV retroviruses (e.g., Human T-lymphotrophic virus type 1 (Attal et al. (1996) EEES Letters 392:220-4); and the Mammalian type C retoviral family (e.g., Moloney murine leukemia virus (Vagner et al. (1995) J. Biol. Chem 270:20316-83), Friend murine leukemia virus, Harvey murine sarcoma virus, Avian retriculoendotheliosis virus (Lopez-Lastra et al. (1997) Hum. Gene Ther 5:1855-65), Murine leukemia virus (env RNA) (Deffaud et al. (2000) J. Virol. 74:846-50), Rous sarcoma virus (Deffaud et al. (2000) J. Virol. 74:11581-8). Each of these references is herein incorporated by reference.

Eukaryotic mRNAs also contain IRES elements including, for example, BiP (Macejak et al. (1991) Nature 355:91); Antennapedia of Drosophilia (exons d and e) (Oh et al. (1992) Genes and Development 6:1643-1653; c-myc; and, the X-linked inhibitor of apoptosis (XIAP) gene (U.S. Pat. No. 6,171,821).

Various synthetic IRES elements have been generated. See, for example, De Gregorio et al. (1999) EMBO J. 75:4865-74; Owens et al. (2001) PNAS 4:1471-6; and Venkatesan et al. (2001) Molecular and Cellular Biology 21:2826-37. For additional IRES elements known in the art, see, for example, rangueil.inserm.fr/IRESdatabase.

In a specific embodiment, the IRES sequence is derived from encephalomyocarditis virus (ECMV).

As used herein, a reporter protein is any protein that can be specifically detected when expressed (i.e., has a detectable signal when expressed), for example, via its fluorescence or enzyme activity. The plasmid comprises a nucleotide sequence encoding a first reporter protein and a nucleotide sequence encoding a second reporter protein. An open reading frame is fused either to the nucleotide sequence encoding a first reporter protein or to the nucleotide sequence encoding a second reporter protein. In some embodiments, the open reading frame is fused to the nucleotide sequence encoding a first reporter protein. In some embodiments, the open reading frame is fused to the nucleotide sequence encoding a second reporter protein. This allows one to study the expression of the linked open reading frame in response to different stimuli. As used herein, “fused” is intended to mean that the amino acids encoded by the ORF and the reporter protein are joined by peptide bonds to create a contiguous protein sequence. Thus, the reporter protein fused to the open reading frame serves as a marker of the stability of the fused open reading frame. The other reporter protein that is unfused to the open reading frame (and thus does not create a contiguous protein sequence with the amino acids encoded by the ORF) serves as an internal control to normalize for cell number and expression variability.

Typically, the first and second reporter proteins have distinguishable detectable reporter signals. For example, the first and second reporter proteins are enzyme proteins having distinguishable signals generated from their products. In some embodiments, the first and second reporter proteins are bioluminescent proteins that emit light at different wavelengths and/or utilize different substrates. Alternatively, the first and second reporter proteins are fluorescent proteins that fluoresce at different wavelengths.

Many reporter proteins known in the art may be used, including but not limited to bioluminescent proteins, fluorescent reporter proteins, and enzyme proteins such as beta-galactosidase, horse radish peroxidase and alkaline phosphatase that produce specific detectable products. The fluorescent reporter proteins include, for example, green fluorescent protein (GFP), cyan fluorescent protein (CFP), red fluorescent protein (RFP) and yellow fluorescent protein (YFP) as well as modified forms thereof e.g. enhanced GFP (EGFP), enhanced CFP (ECFP), enhanced RFP (ERFP), mCHERRY, and enhanced YEP (EYEP).

Examples of bioluminescent proteins, such as luciferases, including but not limited to renilla luciferase (Rluc), firefly luciferase (FLuc) and NanoLuc, are known in the art (see, for example, Fan, F. and Wood, K., Assay and drug development technologies V5 #1 (2007); Gupta, R. et al Nature Methods V8 #10 (2011); Nano-Glo® Luciferase Assay System (Promega) and en.wikipedia.org/wiki/Bioluminescence.

Other non-limiting examples of reporter proteins are shown below:

Species-specific luciferase specificity, cofactor requirements and physical characteristics.

| | | Size | | | |
|---|---|---|---|---|---|
| Organism | Luciferase | (kDa) | Substrate | Requires | Secreted |
| Photinus pyralis | North American firefly | 61 | D-luciferin | Mg, ATP | No |
| | luciferase | | | | |
| Luciola cruciata | Japanese firefly (Genji-botaru) | 64 | D-luciferin | Mg, ATP | No |
| | luciferase | | | | |
| Luciola italica | Italian firefly Luciferase | 64 | D-luciferin | Mg, ATP | No |
| Luciola lateralis | Japanese firefly (Heike) | 64 | D-luciferin | Mg, ATP | No |
| | luciferase | | | | |
| Luciola mingrelica | East European firefly luciferase | 64 | D-luciferin | Mg, ATP | No |
| Photuris pennsylvanica | Pennsylvania firefly luciferase | 64 | D-luciferin | Mg, ATP | No |
| Pyrophorus | Click beetle luciferase | 64 | D-luciferin | Mg, ATP | No |
| plagiophthalamus | | | | | |
| Phrixothrix hirtus | Railroad worm luciferase | 64 | D-luciferin | Mg, ATP | No |
| Renilla reriformis | Renilla luciferase | 36 | Coelenterazine | N/A | No |
| | Rluc8 (mutant of Renilla | 36 | Coelenterazine | N/A | No |
| | luciferase) | | | | |
| | Green Renilla luciferase | 36 | Coelenterazine | N/A | No |
| Gaussia princeps | Gaussia luciferase | 20 | Coelenterazine | N/A | Yes |
| | Gaussia-Dura luciferase | 20 | Coelenterazine | N/A | Yes |
| Cypridina noctiluca | Cypridina luciferase | 62 | Vargulin/Cypridina | N/A | Yes |
| | | | lucifenin | | |
| Cypridina hilgenodorfii | Cypridina (Vargula) luciferase | 62 | Vargulin/Cypridina | N/A | Yes |
| | | | lucifenin | | |
| Metridia longe | Metridia luciferase | 23.8 | Coelenterazine | N/A | Yes |
| Oplophorus gracilorostris | OLuc | 19 | Coelenterazine | N/A | Yes |

In some embodiments, the first and second reporter proteins are selected from the group consisting of renilla luciferase (Rluc), firefly luciferase (FLuc) and NanoLuc. In some embodiments, the first and second reporter proteins are selected from the group consisting of green fluorescence protein and red fluorescence protein.

An open reading frame is fused either to the nucleotide sequence encoding a first reporter protein or to the nucleotide sequence encoding a second reporter protein. The open reading frame is fused to the 5′ or to the 3′ end of the nucleotide sequence. As used herein, an open reading frame or ORF refers to a sequence of nucleotides that codes for a contiguous sequence of amino acids. The translated open reading frame may be all or a portion of a gene encoding a protein or polypeptide of interest. The ORF may be derived from an ORFeome of an organism. A complete ORFeome contains nucleic acids that encode all proteins of a given organism. A representative fraction of a full ORFeome is at least 60% of all proteins expressed by the organism. In some embodiments, the organism is a mammal. In some embodiments, the mammal is human.

In some embodiments, the ORF is an oncogene that encodes all or a portion of an oncoprotein. Examples of oncogenes include, but are not limited to, RAS, MYC, SRC, FOS, JUN, MYB, ABL, BCL2, HOX11, HOX11L2, TAL1/SCL, LMO1, LM02, EGFR, MYCN, MDM2, CDK4, GLI1, IGF2, EGFR, FLT3-ITD, TP53, PAX3, PAX7, BCR/ABL, HER2 NEU, FLT3R, FLT3-ITD, TAN1, B-RAF, E2A-PBX1, and NPM-ALK, as well as fusion of members of the PAX and FKHR gene families, WNT, MYC, ERK EGFR, FGFR3, CDH5, KIT, RET, Interferon regulatory factor 4 (IRF4) and TRK. Other exemplary oncogenes are well known in the art and several such examples are described in, for example, The Genetic Basis of Human Cancer (Vogelstein, B. and Kinzler, K. W. eds. McGraw-Hill, New York, N.Y., 1998).

In some embodiments, the ORF is a transcription factor. Some examples of such transcription factors include (but are not limited to) the STAT family (STATs 1, 2, 3, 4, 5a, 5b, and 6), FOS/JUN, NF KB, HIV-TAT, and the E2F family. In some embodiments, the protein of interest is an IKAROS family zinc finger protein. In some embodiments, the protein of interest is IKZF1, IKZF2, IKZF3, IKZF4, or IKZF5. In some embodiments, the protein of interest is IKZF1 or IKZF3.

The nucleotide sequence encoding a reporter protein and the fused ORF are “in frame”, i.e., consecutive triplet codons of a single polynucleotide comprising the nucleotide sequence encoding the reporter protein and the fused open reading frame encode a single continuous amino acid sequence.

Other aspects of the invention provide an isolated transformed host cell comprising a plasmid described herein. The plasmid may be introduced into the host cell using any available technique known in the art. For example, the plasmid may be introduced into the host cell by lipofection, calcium phosphate transfection, DEAE-dextran mediated transfection, electroporation, transduction, sonoporation, infection and optical transfection. Suitable host cells include, but are not limited to, bacterial cells (e.g., E. coli, Bacillus subtilis, and Salmonella typhimurium), yeast cells (e.g., Saccharomyces cerevisiae and Schizosaccharomyces pombe), plant cells (e.g.,Nicotiana tabacum and Gossypium hirsutum), and mammalian cells (e.g., CHO cells, and 3T3 fibroblasts, HEK 293 cells, U-2 OS cells).

Another aspect of the invention provides a DNA plasmid library comprising a plurality of plasmids described herein (i.e., a collection of more than one plasmid). In some embodiments, the open reading frame of each plasmid in the library is different. In some embodiments, the open reading frame of each plasmid in the library is derived from an ORFeome of an organism. In some embodiments, the organism is a mammal. In some embodiments, the mammal is human.

Another aspect of the invention provides a method for identifying proteins whose levels are modulated by a compound of interest. The method comprises i) contacting host cells transformed with the plasmid library described herein with a compound of interest; (ii) determining ratios of fused reporter protein signal to unfused reporter protein signal in presence and absence of the compound ; (iii) identifying open reading frames that have increased levels when the ratio of fused reporter protein signal to unfused reporter protein signal in the presence of the compound is increased as compared to the ratio of fused reporter protein signal to unfused reporter protein signal in the absence of the compound and identifying open reading frames that have decreased levels when the ratio of fused reporter protein signal to unfused reporter protein signal in the presence of the compound is decreased as compared to the ratio of fused reporter protein signal to unfused reporter protein signal in the absence of the compound.

The host cells may be transformed with the plasmids of the plasmid library using any available technique known in the art. For example, the plasmids may be introduced into the host cell by lipofection, calcium phosphate transfection, DEAE-dextran mediated transfection, electroporation, transduction, sonoporation, optical transfection, or injection.

As used herein, “fused reporter protein signal” refers to the detectable signal of the reporter protein encoded by the nucleotide sequence that is fused to the ORF. As used herein, “unfused reporter protein signal” refers to the detectable signal of the reporter protein encoded by the nucleotide sequence that is not fused to the ORF. In some embodiments, the open reading frame is fused to the nucleotide sequence encoding a first reporter protein. In such embodiments, ratios of first reporter protein signal to second reporter protein signal are determined in presence and absence of the compound. Open reading frames are identified that have increased levels when the ratio of first reporter protein signal to second reporter protein signal in the presence of the compound is increased as compared to the ratio of first reporter protein signal to second reporter protein signal in the absence of the compound, and open reading frames are identified that have decreased levels when the ratio of first reporter protein signal to second reporter protein signal in the presence of the compound is decreased as compared to the ratio of first reporter protein signal to second reporter protein signal in the absence of the compound.

In some embodiments, the open reading frame is fused to the nucleotide sequence encoding a second reporter protein. In such embodiments, ratios of second reporter protein signal to first reporter protein signal are determined in presence and absence of the compound. Open reading frames are identified that have increased levels when the ratio of second reporter protein signal to first reporter protein signal in the presence of the compound is increased as compared to the ratio of second reporter protein signal to first reporter protein signal in the absence of the compound, and open reading frames are identified that have decreased levels when the ratio of second reporter protein signal to first reporter protein signal in the presence of the compound is decreased as compared to the ratio of second reporter protein signal to first reporter protein signal in the absence of the compound.

The compound of interest can be any compound that is known to have or suspected of having a desirable disease modifying activity (e.g., anti-neoplastic activity, anti-apoptotic activity, and anti-inflammatory activity). These include, among others, small organic molecules, macrocylic compounds, nucleotides (including siRNAs, shRNAs), nucleic acids (including vectors capable of inducing gene editing (CRISPR)), peptides, proteins, and carbohydrates. By identifying proteins whose levels are modulated by a compound of interest, the method described herein allows one to evaluate and understand the mechanism underlying the compound's pharmacological activity. In addition, the method described herein allows for discovery of proteins that can be used to monitor, and/or image the pharmacodynamic effects of the compound.

In some embodiments, the compound of interest is an IMiD® (Celegene). IMiDs® compounds are small molecule, orally available compounds that modulate the immune system and other biological targets through multiple mechanisms of action. Examples of IMiDs compounds include, but are not limited to lenalidomide and CC-4047 (pomalidomide). In some embodiments, the compound of interest is a CDK4 inhibitor (see, for example, WO 2002/051849, WO 2000/012496, WO 2011/101417), NEDD8 inhibitors (see, for example, WO 2013/028832) or proteasome inhibitor MG132.

In some embodiments, contacting the host cells transformed with the plasmid library described herein with a compound of interest comprises growing the transformed host cells in the presence of the compound for an appropriate time under suitable culture conditions. Suitable culture conditions, including the duration of the culture, will vary depending on the cell being cultured. However, one skilled in the art can easily determine the culture conditions by following standard protocols, such as those described in the series Methods in Microbiology, Academic Press Inc. Typically, the cell culture medium may contain any of the following nutrients in appropriate amounts and combinations: salt(s), buffer(s), amino acids, glucose or other sugar(s), antibiotics, serum or serum replacement, and other components such as, but not limited to, peptide growth factors, cofactors, and trace elements. In some embodiments, the transfected host cells are grown in the presence of the compound for 15 mins, 30 mins, 1 hour, 2 hours, 4 hours, 6 hours, 8 hours, 10 hours, 12 hours, 14 hours, 16 hours, 18 hours, 20 hours, 24 hours, 30 hours, 48 hours, or 72 hours.

The fused and unfused reporter protein signals in the presence and absence of the compound are determined using methods known in the art. Detectors such as, but not limited to, luminometers, spectrophotometers, and fluorimeters, or any other device that can detect changes in reporter protein activity can be used. Assay systems known in the art that allow for quantitation of a stable reporter signal from two reporter genes in a single sample can be used. Examples include, but are not limited to, Dual-Glo® Luciferase Assay System (Promega) that measures the activities of firefly and Renilla luciferases sequentially from a single sample.

Upon detecting signals generated by the reporter proteins, it is determined whether the compound of interest increases or decreases the expression of the ORF fused to the reporter protein. Such a determination can be carried out by comparing the ratio of the fused reporter protein signal to unfused reporter protein signal in the presence of the compound to the ratio of the fused reporter protein signal to unfused reporter protein signal in the absence of the compound. When the ratio of fused reporter protein signal to unfused reporter protein signal in the presence of the compound is increased as compared to the ratio of fused reporter protein signal to unfused reporter protein signal in the absence of the compound, the ORF is identified as having increased levels (i.e., greater stability) in the presence of the compound of interest. In contrast, when the ratio of fused reporter protein signal to unfused reporter protein signal in the presence of the compound is decreased as compared to the ratio of fused reporter protein signal to unfused reporter protein signal in the absence of the compound, the ORF is identified as having decreased levels (i.e., less stability) in the presence of the compound of interest.

Another aspect of the invention relates to a method for monitoring treatment of a subject with an IMiD compound. The method comprises determining in a sample of a subject treated with an IMiD compound a level of IKZF1 and/or IKZF3; and identifying the subject as responding to the treatment when the level of IKZF1 and/or IKZF3 is decreased as compared to a reference level.

The term “subject,” as used herein, refers to an mammal who has a condition or disorder that is being treated with an IMiD compound. Examples of conditions or disorder that can be treated with IMiDs include, but are not limited to, cancers such as multiple myeloma and myelofibrosis, transfusion-dependent anemia, and myelodysplastic disorders. In some embodiments, the subject is a human. In some embodiments, the subject is a non-human mammal. In some embodiments, the subject is a non-human primate. In some embodiments, the subject is a sheep, a goat, a cattle, a cat, or a dog. Examples of IMiDs compounds include, but are not limited to lenalidomide and CC-4047 (pomalidomide). “Monitoring treatment of a subject with an IMiD compound” refers to ascertaining the progression or remission of the condition or disorder being treated with an IMiD compound. In some embodiments, “monitoring treatment of a subject with an IMiD compound” refers to ascertaining whether the IMiD compound is having its predicted and desired pharmaceutical effect.

As used herein, a “sample obtained from a subject” refers to a sample of tissue or fluid isolated from a subject, including but not limited to, for example, blood, plasma, serum, fecal matter, urine, bone marrow, bile, spinal fluid, lymph fluid, samples of the skin, external secretions of the skin, respiratory, intestinal, and genitourinary tracts, tears, saliva, milk, blood cells, organs, and biopsies. In some embodiments, the sample is blood, plasma or tumor tissue.

Obtaining a sample of a subject means taking possession of a sample of the subject. Obtaining a sample from a subject means removing a sample from the subject. Therefore, the person obtaining a sample of a subject and determining a level of IKZF1 and/or IKZF3 in the sample does not necessarily obtain the biological sample from the subject. In some embodiments, the sample may be removed from the subject by a medical practitioner (e.g., a doctor, nurse, or a clinical laboratory practitioner), and then provided to the person determining a level of IKZF1 and/or IKZF3. The sample may be provided to the person determining a level of IKZF1 and/or IKZF3 by the subject or by a medical practitioner (e.g., a doctor, nurse, or a clinical laboratory practitioner). In some embodiments, the person determining a level of IKZF1 and/or IKZF3 obtains a biological sample from the subject by removing the sample from the subject. It is to be understood that sample may be processed in any appropriate manner to facilitate measuring a level of IKZF1 and/or IKZF3. For example, biochemical, mechanical and/or thermal processing methods may be appropriately used to isolate a biomolecule of interest from a biological sample. The level of IKZF1 and/or IKZF3 may also be determined in a sample directly.

IKZF1 (IKAROS Family Zinc Finger 1 (Ikaros); Gene ID: 10320) encodes a transcription factor that belongs to the family of zinc-finger DNA binding proteins associated with chromatin remodeling. It displays crucial functions in the hematopoietic system and its loss of function has been linked to the development of lymphoid leukemia. In particular, Ikaros has been found in recent years to be a major tumor suppressor involved in human B-cell acute lymphoblastic leukemia.

IKZF3 (Ikaros family zinc finger protein 3; Gene ID: 22806) is a transcription factor that is important in the regulation of B lymphocyte proliferation and differentiation. It is involved in regulating BCL2 expression and controlling apoptosis in T-cells in an IL2-dependent manner.

As used herein, “determining a level of IKZF1 and/or IKZF3” refers to determining the amount or concentration of IKZF1 and/or IKZF3 in the sample. “Determining” refers to performing an assay to measure the level of IKZF1 and/or IKZF3. In some embodiments, “determining” includes, for example, determining the expression level or activity level of IKZF1 and/or IKZF3 in the sample. In some embodiments, the expression level of the IKZF1 and/or IKZF3 protein is determined.

The level of IKZF1 and/or IKZF3 may be measured by performing an assay. “Performing an assay” means testing a sample to quantify a level of IKZF1 and/or IKZF3. Examples of assays used include, but are not limited to, mass spectroscopy, gas chromatography (GC-MS), HPLC liquid chromatography (LC-MS), and immunoassays. In some embodiments, the level of IKZF1 and/or IKZF3 is determined by measuring the level a reporter protein fused to IKZF1 or IKZF3 (see Zhang et al. Nature Medicine 10, 643-648 (2004) and Safran et al. Proc Natl Acad Sci USA. 2006 Jan. 3;103(1):105-10). Other appropriate methods for determining a level of IKZF1 and/or IKZF3 will be apparent to the skilled artisan.

The subject is identified as responding to the treatment when the level of IKZF1 and/or IKZF3 is decreased as compared to a reference level. The reference level is the level of IKZF1 and/or IKZF3 in a control subject that has not been treated with the IMiD compound.

According to some aspects, the present disclosure provides a method to characterize function of a protein of interest. The method comprises providing a cell having a protein of interest fused to IKZ1 or IKZ3 or fragments thereof; contacting the cell with an IMiD compound; and monitoring the effects of down regulating the protein of interest. In some embodiments, the cell is in vivo.

As used herein, a “protein of interest” can be any conceivable polypeptide or protein that may be of interest, such as to study or otherwise characterize. In some embodiments, the protein of interest is a human polypeptide or protein. In some embodiments, the protein of interest is an oncoprotein, such as, but not limited to, RAS, MYC, SRC, FOS, JUN, MYB, ABL, BCL2, HOX11, HOX11L2, TAL1l/SCL, LMO1, LM02, EGFR, MYCN, MDM2, CDK4, GLI1, IGF2, EGFR, FLT3-ITD, TP53, PAX3, PAX7, BCR/ABL, HER2 NEU, FLT3R, FLT3-ITD, TAN1, B-RAF, E2A-PBX1 , and NPM-ALK, as well as fusion of members of the PAX and FKHR gene families, WNT, MYC, ERK EGFR, FGFR3, CDH5, KIT, RET, and TRK. Other exemplary oncogenes are well known in the art and several such examples are described in, for example, The Genetic Basis of Human Cancer (Vogelstein, B. and Kinzler, K. W. eds. McGraw-Hill, New York, N.Y., 1998).

In some embodiments, the protein of interest is a transcription factor. Some examples of such transcription factors include (but are not limited to) the STAT family (STATs 1, 2, 3, 4, 5a, 5b, and 6), FOS/JUN, NF KB, HIV-TAT, and the E2F family. In some embodiments, the protein of interest is an IKAROS family zinc finger protein.

As used herein a “fragment” of IKZF1 or IKZF3 refers to any portion of IKZF1 or IKZF3 smaller than the corresponding full-length protein. The fragment includes the amino acids sufficient for binding to cereblon in the presence of an IMiD.

In some embodiments, monitoring the effects of down regulating the protein of interest refers to monitoring the viability of the cell using any method known in the art.

The present invention is further illustrated by the following Example, which in no way should be construed as further limiting. The entire contents of all of the references (including literature references, issued patents, published patent applications, and co-pending patent applications) cited throughout this application are hereby expressly incorporated by reference.

### EXAMPLE

### Materials and Methods

Cell culture

HEK 293FT (Invitrogen) cells were maintained in DMEM medium supplemented with 10% fetal bovine serum, 100 U/ml penicillin, and 100 μg/ml streptomycin. U937, MM1S, KMS34, KMS11, L363, RPMI8226 and OCImy5 cells were cultured in RPMI medium containing 10% fetal bovine serum, 100 U/ml penicillin, and 100 μg/ml streptomycin. Stable cell lines were established by lentiviral infection followed by fluorescence activated cell sorting or growth in media containing 0.5 or 2 μg/ml puromycin, 10 μg/ml blasticidin, or 200 μg/ml hygromycin.

### Luciferase ORF Fusion Library Construction

The destination vector pCMV-IRES-Renilla Luciferase-IRES-Gateway-Firefly Luciferase (pIRIGF) was constructed by overlapping PCR. The human ORFeome library V5.1 was shuttled into pIRIGF via LR gateway recombination (Invitrogen). After recombination overnight at 25° C., 1 μL of reaction mix was transformed into 10 μL of OmniMAX 2T1 competent cells (Invitrogen) in 96-well plate through heat shock at 42° C. for 1 minute. 100 μL of SOC media were added into each well and the transformation mixtures were incubated at 37° C. for 1 hour. Next, each mixture was transferred into 1 mL 2x YT media containing 100 μg/mL ampicillin using 96-well deep well plates (Qiagen). After shaking overnight at 37° C., transformants were collected by centrifugation and plasmids were extracted using the DirectPrep 96 BioRobot Kit (Qiagen).

### 384-Well Dual-Glo Luciferase Screen

To transfect the ORF luciferase fusion library into 293FT cells seeded in 384-well plates, 25 μl of media containing 7000 cells was plated about 10 minutes prior to adding the DNA/transfection mixture. Using a Beckman FX pod head of 96-disposable tips, individual DNA plasmid solutions (5 μl) arrayed according to the original ORFeome V5.1 map (169 library plates) were diluted into 20 μl of Opti-MEM reduced serum media. 20 μl of diluted plasmid was then mixed with 20 μl of diluted Lipofectamine 2000 (Invitrogen) [1:15, diluted in Opti-MEM reduced serum media]. After 5 minutes at room temperature, 3 μl of this DNA/lipofectamine solution was added into 4 well quadrants of two identical 384-well plates (total of 8 wells) corresponding to the single 96-well source solution. The cells were then grown for 26-28 hours under standard growth conditions. An 8-channel multi-drop dispenser was used to add 5 μl of a 13.3 μM solution of lenalidomide (final 2 μM; Selleck Chemicals #S1029) to one plate and 0.13% DMSO to the other plate and the cells were allowed to grow for an additional 17-20 hours. Expression of firefly and renilla luciferase were quantified using the Dual-glo assay according to manufacturer's instructions (Promega #E2940). For data analysis, the raw counts per second (CPS) were first normalized for total transfection efficiency by dividing the firefly CPS by renilla CPS (Fluc/Rluc) from each well. Then the four replicate well ratios on each plate were averaged and the standard deviation was determined. The lenalidomide (LEN) induced changes were then determined for each ORF by dividing the averaged Fluc/Rluc ratio from the LEN plate by the DMSO control plate (change of Fluc/Rluc). Data associated with all significant changes in ratio were confirmed to originate from raw data significantly above background signal. Any quadruplicate data that had >50% standard deviation was eliminated from further analysis in order to quickly remove most of the data from low expression plasmids and otherwise error prone data. Most of the excluded data were from low expressing clones. The remaining data (13,372 out of 15,483 ORF's) were then fit to a log normal distribution with mean LEN/DMSO ratio of 1.02±0.18, and a list of ORF whose ratio was increased (61) or decreased (46) by 3 standard deviations was generated for follow-up testing.

### 96-Well Dual-Glo Luciferase Assay

To verify the change of Fluc/Rluc of ORFs obtained from the 384-well high-throughput screen, each clone was further tested in a 96 well format. The day before transfection, 293FT cells were seeded into solid opaque 96-well plates (BD biosciences) with 20,000 cells in 100 μl of DMEM culture media per well. On the day of transfection 96-well polypropylene plates (Greiner) were used to prepare the transfection mixture. First, a plasmid mixture containing 240 ng of ORF luciferase fusion clone and 480 ng of pcDNA3 was diluted into 120 μl of Opti-MEM reduced serum. Next 14.4 μL of Lipofectamine 2000 was diluted into 120 μl of Opti-MEM reduced serum. 5 minutes later, the plasmid solution was mixed with the diluted Lipofectamine 2000 using a multi-channel pipette. After 30 minutes, 28 μl of DNA/lipofectamine mixture was added to each well of the 96 well plates seeded with 293FT cells (8 wells for each ORF). 24 hours after transfection, cells were treated with LEN (2 μM) or DMSO in quadruplicate. 36 hours after transfection, the firefly and renilla luciferase signals were quantified using the Dual-glo assay according to manufacturer's instructions. The ratio of FLuc/Rluc was calculated for each well. To determine the change of Fluc/Rluc for each ORF, the averaged Fluc/Rluc of LEN treated wells was divided by that of DMSO treated wells. A 2-tailed Student's t test was performed to determine the statistical significance. P value <0.05 was considered significant.

### Plasmids

Human IKZF4 cDNA clone was PCR amplified from ETS clone HsCD00295530 (PlasmID, DF/HCC DNA Resource Core), and then cloned into pDONR223 via BP gateway recombination. Human CRBN, human IKZF1 splicing variant 2 (IKZF1-V2), human IKZF2 splicing variant 2 (IKZF2-V2), human IKZF5 and human IRF4 in pDONR223 entry vectors were obtained from the human ORFeome Collection (DF/HCC DNA Resource Core). IKZF1 splicing variant 1 (IKZF1-V1) cDNA and human IKZF2 splicing variant 1 (IKZF2-V1) cDNA were generated by overlapping PCR and cloned into pDONR223 via BP gateway recombination. IKZF4, IKZF1-V1 and IKZF2-V1 were cloned into pIRIGF via LR gateway recombination. IKZF1/2 chimeras (IKZF12 H1, IKZF12 H2, IKZF12 H3, IKZF12 H4, IKZF21 H5, IKZF21 H6, IKZF21 H7, IKZF2 H8, IKZF121 and IKZF212), IKZF1-V2-Q146H, IKZF1-V2-H176P/L177F, IKZF2-V1-H141Q, IKZF2-V1-P171H/F172L, IKZF1-V1-Q146H, IKZF3-Q147H mutants were generated via overlapping PCR and cloned into pIRIGF via LR gateway recombination. IKZF1-V2, IKZF2-V2, IKZF3, IKZF4, IKZF5 and IRF4 were cloned into plenti-UBC-gate-3xHA-pGK-PUR via LR gateway recombination. IKZF1-V2, IKZF121, IKZF1-V2-Q146H, IKZF1-V2-H176P/L177F, IKZF2-V1-H141Q, IKZF2-V1-P171H/F172L, IKZF212, IKZF1-V1, IKZF1-V1-Q146H, IKZF3 and IKZF3-Q147H were cloned into plenti-UBC-gate-3xHA-pGK-HYG. IKZF1-V2 and IKZF2-V2 were cloned into pcDNA3.2-DEST (Invitrogen) and plenti-UBC-gate-FLAG-pGK-HYG via LR gateway recombination. IKZF1-V1, IKZF1-V1-Q146H, IKZF3, IKZF3-Q147H, IKZF2-V2 and IRF4 were cloned into plenti-CAG-gate-FLAG-IRES-GFP via LR recombination. CRBN-Y383A/W385A (YWAA) mutant was generated by overlapping PCR and cloned into pDON223 via BP recombination. CRBN and YWAA were cloned into pcDNA3-FLAG-gate-pGK-HYG, plenti-UBC-FLAG-gate-pGK-HYG, plenti-UBC-HA-gate-pGK-HYG, and plenti-UBC-Myc-gate-pGK-HYG via LR recombination. The cDNAs within the lentiviral expression vectors were confirmed by DNA sequencing.

### CRISPR Genome Editing

The CRBN gene editing vectors were generated by cloning the annealed oligonucleotide pair into pX330 digested with BbsI (16) (CRBN T1 Forward 5′-CACCGTCCTGCTGATCTCCTTCGC-3′ (SEQ ID NO: 1), CRBN T1 Reverse 5′-AAACGCGAAGGAGATCAGCAGGAC-3′ (SEQ ID NO: 2); CRBN T2 Forward 5′-CACCGAAACAGACATGGCCGGCGA-3′(SEQ ID NO: 3), CRBN T2 Reverse 5′-AAACTCGCCGGCCATGTCTGTTTC-3′(SEQ ID NO: 4)). 293FT cells seeded in a 12-well plate were transiently transfected with 1 μg of CRBN pX330 editing vector using Lipofectamine 2000. Three days after transfection, cells were trypsinized, placed in fresh growth media, and seeded into 96 well plates via serial dilution to obtain single clones. Two weeks later, single clones were picked and expanded to verify the editing of CRBN by immunoblot analysis. To target MM1S cells, the CRBN pX330 editing vector was shuttled into a lentirviral backbone containing a pGK-Pur cassette. MM1S cells were then infected with plenti-CRBN CRISPR pGK-pur and selected with 0.5 μg/mL puromycin for three days. Stable cells were plated in 96 well plates via limiting dilution. Four weeks later, single clones were expanded and CRBN−/− clones were identified by immunoblot analysis.

### Immunoblotting

Cells were washed twice with ice cold PBS and harvested in Buffer A [50 mM Tris (pH 7.4), 150 mM NaCl, 1% Triton X-100, 1 mM EDTA, 1 mM EGTA, 1 mM β-glycerophosphate, 2.5 mM sodium pyrophosphate, 1 mM Na3VO4, 10 mM NaF, and 1x protease inhibitor (Roche)]. Whole cell extracts were resolved by SDS-polyacrylamide gel electrophoresis, transferred onto a nitrocellulose membrane, and probed with the indicated primary antibodies. Bound antibodies were detected with horseradish peroxidase-conjugated secondary antibodies (Pierce) and enhanced chemiluminescence (ECL) western blotting detection regents (Pierce) or Immobilon western chemiluminescent horseradish peroxidase substrate (Millipore).

### Antibodies

The following antibodies were used: HRP conjugated anti-FLAG M2 mouse monoclonal antibody (Sigma), HRP conjugated anti-HA mouse monoclonal antibody (Cell signaling), HRP conjugated anti-Myc mouse monoclonal antibody (Cell Signaling), FLAG M2 mouse monoclonal antibody (Sigma), CRBN rabbt polyclonal antibody (Novus Biologicals), IKZF1 rabbit polyclonal antibody (Cell Signaling, for immunoblot), IKZF1 rabbit polyclonal antibody (Bethyl, for chromatin immunoprecipitation), IKZF2 rabbit polyclonal antibody (Bethyl), IKZF3 rabbit polyclonal antibody (Imginex), HA.11 mouse monoclonal antibody (Covance), Vinculin mouse monoclonal antibody (Sigma), DDB1 rabbit polyclonal antibody (Cell Signaling), IRF4 rabbit polyclonal antibody (Cell Signaling), Cul4A rabbit polyclonal antibody (Cell Signaling), Goat anti-rabbit HRP conjugated antibody (Thermo Scientific), Goat anti-mouse HRP conjugated antibody (Thermo Scientific).

### Immunoprecipitation

Cells were lysed in Buffer B [50 mM Tris (pH 7.4), 150 mM NaCl, 0.5% NP-40, 1 mM β-glycerophosphate, 2.5 mM sodium pyrophosphate 1 mM Na3VO4, 10 mM NaF, and 1x protease inhibitor (Roche)]. The lysates were clarified by centrifugation and then mixed with primary antibody for 12 hours at 4° C. Where indicated, LEN (2 μM) or DMSO were used to treat the cells prior to cell lysis (Pre), or added into whole cell extracts after cell lysis (Post). Immune complexes were captured with protein G agarose beads (Roche) for 1 hour at 4° C. and then washed 6 times with Buffer B. For anti-FLAG immunoprecipitation, cell extracts were pre-cleared with protein G agarose beads at 4° C. for 1 hour and then incubated with anti-FLAG M2 agarose beads at 4° C. for 6 hours, followed by six washes with Buffer B. Bound proteins were eluted by boiling in SDS loading buffer and detected by immunoblot analysis.

### Postlysis Binding Assays (Mixing Experiments)

293FT CRBN−− T11 (CRIPSR T1 clone 1) cells infected with lentiviral vectors expressing FLAG-tagged IKZF1 or FLAG-tagged IKZF2, or empty lentiviral vectors were treated with DMSO or LEN (2 μM) for 12 hour. Cells were then washed with ice-cold PBS twice and lysed in Buffer B. Total cell extract was collected after centrifugation and incubated with anti-FLAG M2 agarose beads at 4° C. overnight. Next, the beads were washed with Buffer B four times. The IKZF1 or IKZF2 loaded beads were then mixed with whole cell lysates prepared from 293FT CRBN+/+ cells treated with DMSO or LEN (2 μM) for 12 hour prior to cell lysis (Pre). After binding overnight at 4° C., the agarose was washed six times with Buffer B. Bound proteins were then eluted by boiling in SDS loading buffer, resolved by SDS-PAGE, and detected by immunoblot analysis. The binding of FLAG-tagged IKZF1 with endogenous cereblon was also assayed using 293FT cell extracts that were treated with LEN (2 μM) or DMSO post cell lysis (Post).

To assay binding of FLAG-tagged IKZF1 to exogenous cereblon, the IKZF1 loaded anti-FLAG M2 agarose beads were washed four times with Buffer B and then mixed with whole cell extracts prepared from 293FT CRBN null T11 cells (transiently transfected with HA-tagged cereblon (CRBN) or cereblon mutant (YWAA) and treated with DMSO or LEN (2 μM) for 12 hour before lysis). After binding overnight at 4° C., the agarose was washed six times with Buffer B. Bound proteins were then eluted by boiling in SDS loading buffer, resolved by SDS-PAGE, and detected by immunoblot analysis.

To assay binding of recombinant cereblon with endogenous IKZF1 and IKZF3, FLAG-tagged CRBN or YWAA mutant transiently expressed in 293FT CRBN−/− T11 cells was immunoprecipitated using anti-FLAG M2 agarose beads. The cereblon loading beads were washed four times with Buffer B and then mixed with MM1S CRBN−/− T11 cell extracts that were treated with LEN (10 μM) or DMSO after cell lysis. After binding overnight at 4° C., the agarose was washed six times with Buffer B. Bound proteins were then eluted by boiling in SDS loading buffer, resolved by SDS-PAGE, and detected by immunoblot analysis.

### In Vitro Binding and Ubiquitylation Assay

293FT CRBN null T11 cells were transiently transfected with plasmids encoding Myc-tagged cereblon (CRBN) or cereblon mutant (YWAA), HA-tagged IKZF1, or with empty vector (EV). 48 hours later, the cells were washed twice with ice cold PBS and lysed in Buffer B. Myc-CRBN or Myc-YWAA cell extracts were mixed with HA-IKZF1 extract in the absence of presence of LEN (2 μM) or DMSO. Anti-Myc antibody conjugated agarose beads (Sigma) were then added to mixed extracts, following by incubation at 4° C. for 12 hours. As a negative control, 5% of the HA-IKZF1 extract used to mix with CRBN extract was mixed with extracts from the empty vector cells and then incubated with anti-HA antibody conjugated beads (Sigma). Next, the beads were washed four times with buffer B, twice with 1xURB buffer [50 mM Tris (pH 7.4), 5 mM KCl, 5 mM NaF, 5 mM MgCl2, 0.5 mM DTT], and then resuspended in 20 μL of 1x URB buffer containing 200 ng recombinant E1 (Boston Biochem), 250 ng recombinant UbcH5a (Boston Biochem), 250 ng recombinant UbcH5b (Boston Biochem), 0.4 μM Ubiquitin aldehyde (Boston Biochem), 10 μg FLAG Ubiquitin (Boston Biochem), 1x ERS (20 mM creatine phosphate, 0.2 μg/μl creatine phosphokinase, 5 mM Mg-ATP), 1x Protease/phosphatase inhibitor (Roche), 10 nM LLNL (Boston Biochem), 10 nM MG132 (EMD Bioscience), 0.5 mM ATP. The reaction was carried out at 30° C. for 1 hour and then terminated by boiling in SDS loading buffer. Ubiquitylation products were resolved by SDS-PAGE and detected by immunoblot analysis.

### In Vivo Ubiquitylation Assay

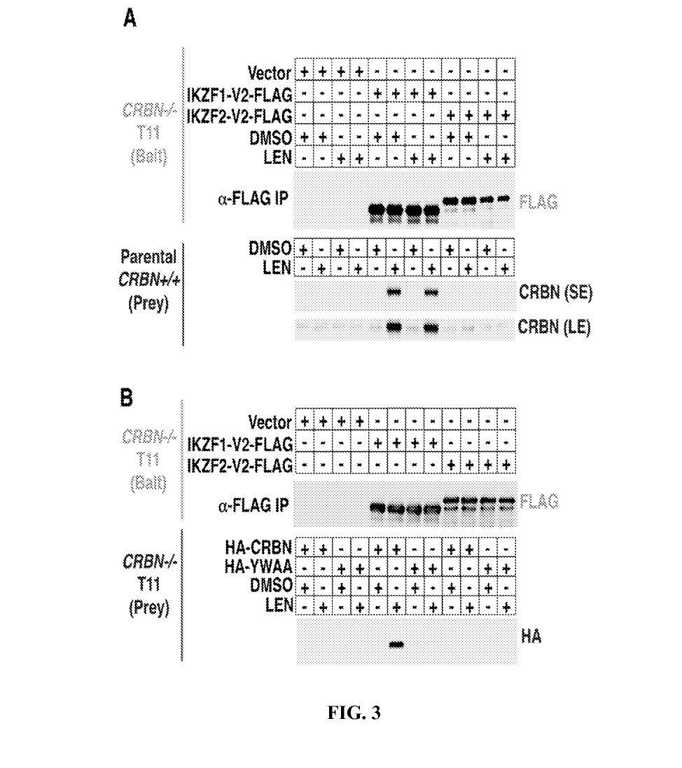

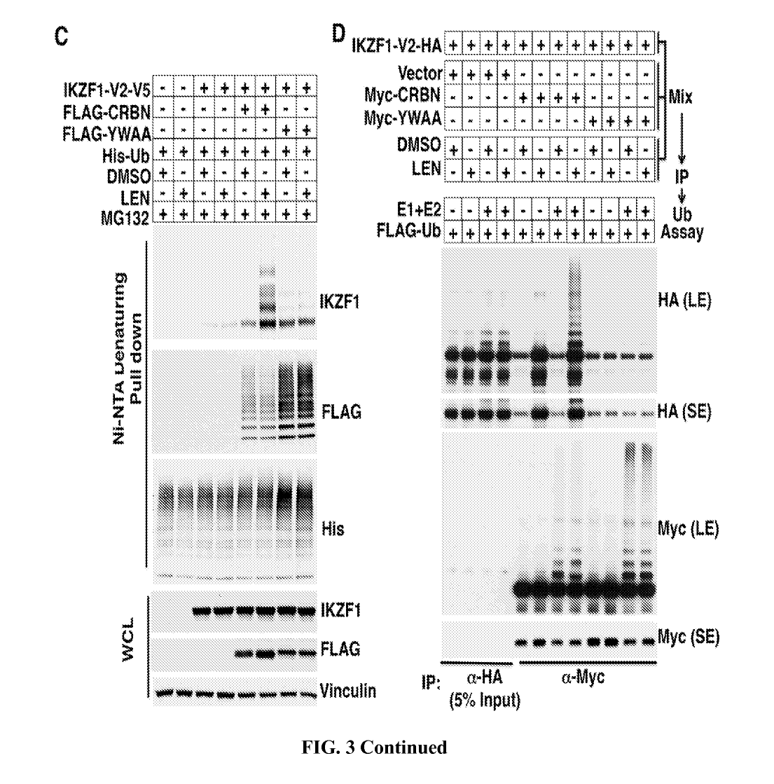

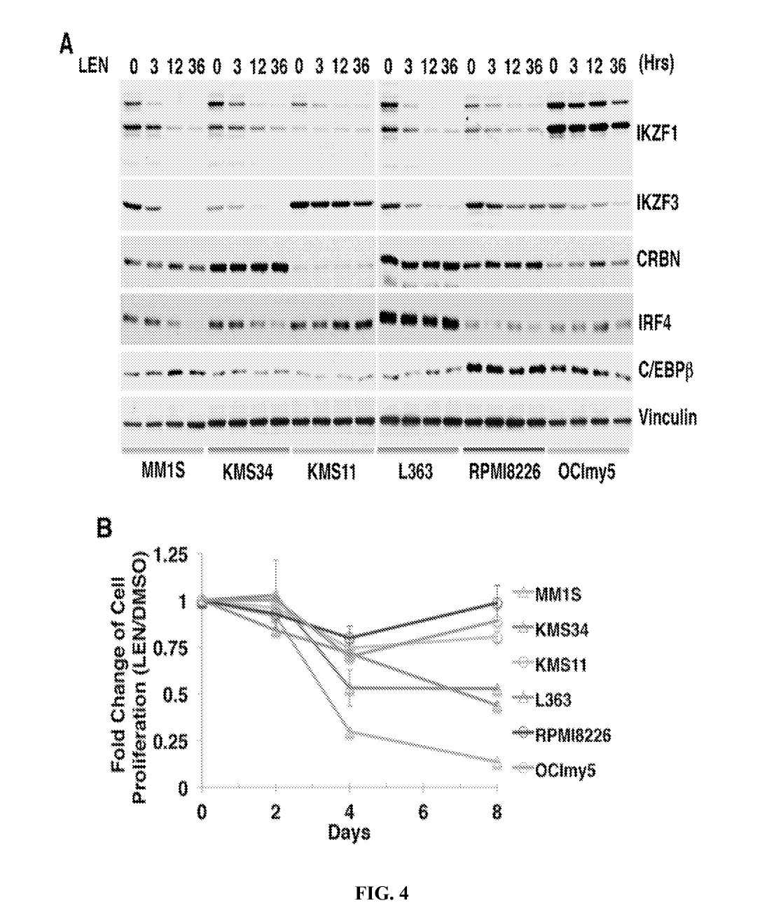

The ubiquitylation assays were carried out as described previously (30). In brief, 293FT cells seeded in 6 well plates were transiently transfected with the following plasmids as indicated in FIG. 3C: pcDNA3-IKZF1-V2-V5 (1 μg), pcDNA3-FLAG-CRBN (1 μg), pcDNA3-FLAG-YWAA (1 μg) and pCMV-8x His-Ub (0.5 μg). Empty vector pcDNA3 was used to bring the total plasmid DNA to 4 μg for each transfection. Thirty-six hours later, cells were treated with LEN (2 μM) or DMSO as well as 10 μM MG132 for additional 12 hours. The cells were then washed twice with ice cold PBS, scraped off the plates in PBS and then collected by centrifugation. A small aliquot of the cells was lysed in Buffer A, and the rest were lysed in Buffer C (6M guanidine-HCL, 0.1M Na2HPO4/NaH2PO4, 10 mM imidazole, pH 8.0). After sonication, the whole cell extract were mixed with 25 μL of Ni-NTA agarose beads at room temperature for 3 hours. Next, the Ni-NTA beads were washed twice with Buffer C, twice with Buffer D (1 Volume of Buffer C: 3 volumes of Buffer E), and once with Buffer E (25 mM Tris.CL, 20 mM imidazole, pH 6.8). Bound proteins were then eluted by boiling in 1SDS loading buffer containing 300 mM imidazole, resolved by SDS polyacrylamide gel electrophoresis, and detected by immunoblot analysis.

### In Vivo Protein Degradation Assay

293FT cells seeded in 12-well plates were transiently transfected with 50 ng of plenti-UBC-pGK-HYG or plenti-UBC-pGK-Pur lentiviral vectors expressing the indicated ORFs and 950 ng of the corresponding empty lentiviral vector. Thirty-six hours later, cells were treated with DMSO, LEN (2 μM) or POM (0.2 μM) for 12 hours. Cell extracts were harvested using Buffer A and subjected to immunoblot analysis.

### Protein Half Life Analysis

For cycloheximide assays, cells were pretreated with DMSO or LEN (2 μM) for 1 hour, and then 100 μg/ml cycloheximide (Sigma) was added into the DMEM growth medium. At various time points thereafter cell extracts were harvested using Buffer A and subjected to immunoblot analysis.

### Realtime RT-PCR

Total RNA was extracted using Qiagen RNeasy Mini Kit with on-column DNase digestion. Total RNA was reverse transcribed into first-strand cDNA using AffinityScript QPCR cDNA Synthesis Kit (Agilent) with random primers. Real-time PCR was performed in duplicate using RT2 SYBR Green/ROX qPCR Master Mix (Qiagen) and the Mx3000P QPCR system (Stratagene). Values were averaged to calculate the expression level and normalized against the level of RPS18. PCR primers are as follows:

| (SEQ ID NO: 5) | |
|---|---|
| RPS18 For, | 5′-TTCGGAACTGAGGCCATGAT-3′ |
| (SEQ ID NO: 6) | |
| RPS18 Rev, | 5′-TTTCGCTCTGGTCCGTCTTG-3′ |
| (SEQ ID NO: 7) | |
| IKZF1 For, | 5′-CCCCTGTAAGCGATACTCCA-3′ |
| (SEQ ID NO: 8) | |
| IKZF1 Rev, | 5′-TGGGAGCCATTCATTTTCTC-3′ |
| (SEQ ID NO: 9) | |
| IKZF3 For, | 5′-TCGGAGATGGTTCCAGTTATCA-3′ |
| (SEQ ID NO: 10) | |
| IKZF3 Rev, | 5′-ATTCTGGCGTTCTTCATGGTT-3′ |
| (SEQ ID NO: 11) | |
| IRF4 For, | 5′-GCGGTGCGCTTTGAACAAG-3′ |
| (SEQ ID NO: 12) | |
| IRF4 Rev, | 5′-ACACTTTGTACGGGTCTGAGA-3′ |

Lentiviral shRNA Vectors

Synthetic complementary oligonucleotides targeting the mRNA of interest were annealed and subcloned as follows: Oligonucleotides targeting CRBN or a control oligonucleotide pair (shCNTL) ligated into pLKO-tet-on-puro (31). Oligonucleotides targeting IKZF1 and IKZF3 and shCNTL were ligated into pLKO-RFP (a gift from Dr. Julie Losman). Targeting sequences are listed as follows:

| (SEQ ID NO: 13) | |
|---|---|
| shCNTL, | 5′-CAACAAGATGAAGAGCACCAA-3′; |
| (SEQ ID NO: 14) | |
| shCRBN-1, | 5′-GCCCACGAATAGTTGTCATTT-3′; |
| (SEQ ID NO: 15) | |
| shCRBN-2, | 5′-GCTTGCAACTTGAATCTGATA-3′; |
| (SEQ ID NO: 16) | |
| shIKZF1-1, | 5′-GCATTTGGAAACGGGAATAAA-3′; |
| (SEQ ID NO: 17) | |
| shIKZF1-2, | 5′-CTACGAGAAGGAGAACGAAAT-3′; |
| (SEQ ID NO: 18) | |
| shIKZF1-3, | 5′-CCGCTTCCACATGAGCTAAAG-3′; |
| (SEQ ID NO: 19) | |
| shIKZF3-1, | 5′-GCCTGAAATCCCTTACAGCTA-3′; |
| (SEQ ID NO: 20) | |
| shIKZF3-2, | 5′-GTAACCTCCTCCGCCACATTA-3′; |
| (SEQ ID NO: 21) | |
| shIKZF3-3, | 5′-GACAGTCTAAGAGTAAGTAAA-3′. |

cDNA Depletion or Enrichment Assay

MM1S cells were infected with plenti-CAG-FLAG-IRES-GFP empty vector or plenti-CAG-FLAG-IRES-GFP vectors expressing IKZF1-V1-Q146H, IKZF3-Q147H, IKZF2-V2 or IRF4. 48 hours after infection, 2 μM LEN or DMSO were added to the culture media. The media was replaced with fresh media containing LEN or DMSO every other day. The percentage of GFP+ cells at the indicated time points thereafter was determined by flow cytometric analysis. The fold change in the percentage of GFP+ cells was calculated by dividing the percentage of LEN treated cells by that of DMSO treated cells.

Small Hairpin RNA (shRNA) Depletion or Enrichment Assay

MM1S or KMS34 cells were infected with pLKO-RFP lentiviral vectors expressing control shRNA or shRNAs against IKZF1 or IKZF3. Two days later the percentage of RFP+cells was monitored using flow cytometer. The change of RFP+ percentage for each shRNA was first normalized against day 2. Then, the relative percentage of RFP+ cells was normalized against shRNA control for each time point as indicated.

### Chromatin Immunoprecipitation

ChIP studies were carried out as described previously with several modifications using specific primers for the human IRF4 locus (32). Briefly, 4x107 MM1.S cells were treated 24 hours with 2 μM LEN or DMSO and crosslinked for 10 minutes at room temperature by the addition of one-tenth of the volume of 11% formaldehyde solution (11% formaldehyde, 50mM HEPES pH 7.3, 100 mM NaCl, 1 mM EDTA pH 8.0, 0.5 mM EGTA pH8.0) to the growth media followed by quenching with 0.125M glycine and two washes with PBS. Fifty μl of Dynal protein 6 magnetic beads (Sigma) were blocked with 0.5% BSA (w/v) in PBS. Magnetic beads were bound with 10 μg of the anti-IKZF1 antibody (Bethyl Labs #A303-516A). Crosslinked cells were lysed with lysis buffer 1 (50 mM HEPES-KOH pH 7.5, 140 mM NaCl, 1 mM EDTA pH 8.0, 10% glycerol, 0.5% NP-40, and 0.25% Triton X-100) and washed with lysis buffer 2 (10 mM Tris-HCl pH 8.0, 200 mM NaCl, 1 mM EDTA pH 8.0, and 0.5 mM EGTA pH 8.0). Cells were resuspended and sonicated in lysis buffer 3 (50 mM HEPES-KOH pH 7.5, 140 mM NaCl, 1 mM EDTA pH 8.0, 1 mM EGTA pH 8.0, 1% Triton X-100, 0.1% Na-Deoxycholate and 1% SDS) for 4×10 minute cycles, 30 second on/off cycles using a Bioruptor sonicator on power setting HIGH. Sonicated lysates were cleared, diluted 1:10 with dilution buffer (50 mM HEPES-KOH pH 7.5, 140 mM NaCl, 1 mM EDTA pH 8.0, 1 mM EGTA pH 8.0, 1% Triton X-100, 0.1% Na-Deoxycholate) and incubated overnight at 4° C. with magnetic beads bound with antibody. Beads were washed two times with lysis buffer 3, once with high salt wash (50 mM HEPES-KOH pH 7.5, 500 mM NaCl, 1 mM EDTA pH 8.0, 1 mM EGTA pH 8.0, 1% Triton X-100, 0.1% Na-Deoxycholate and 0.1% SDS), once with LiCl wash buffer (20 mM Tris-HCl pH 8.0, 1 mM EDTA pH 8.0, 250 mM LiCl, 0.5% NP-40, 0.5% Na-deoxycholate), and once with TE buffer (10 mM Tris-HCl pH 8.0, 1 mM EDTA pH 8.0). Protease inhibitors (Roche Complete) were added to all lysis and wash buffers. Bound complexes were eluted twice in elution buffer (50 mM Tris-HCl pH 8.0, 10 mM EDTA pH 8.0, 1% SDS) at 65° C. for 15 min with occasional vortexing. Crosslinks were reversed overnight at 65° C. RNA and protein were digested using RNase A and Proteinase K, respectively, and DNA was purified with phenol chloroform extraction and ethanol precipitation. Primers were designed to amplify 2 sites within the promoter region, and a negative control region 3′ of the transcribed region: Promoter site 1 (forward) 5′-AGTTGCAGGTTGACCTACGG-3′ (SEQ ID NO: 22) and (reverse) 5′-AGCTTTCACCCGTTGAGCTT-3′ (SEQ ID NO: 23); Promoter site 2 (forward) 5′-ACTCTCAGTTTCACCGCTCG-3′ (SEQ ID NO: 24) and (reverse) 5′-CTCCGGGTCCTCTCTGGTAT-3′ (SEQ ID NO: 25); Negative control region 1 (forward) 5′-CGTGGCTATGTTTGCTTGGG-3′ (SEQ ID NO: 26) and (reverse) 5′-AGCAGGCCTCTTGGTTGTTT-3′ (SEQ ID NO: 27); Negative control region 2 (forward) 5′-GCAGTGCTGACACTGGATCT-3′ (SEQ ID NO: 28) and (reverse) 5′-GCCTGCCATGCGTAATCAAG-3′(SEQ ID NO: 29). Enrichment data were analyzed by calculating the immunoprecipitated DNA percentage of input DNA for each sample.

### Cell Proliferation Assay

1.5×106 viable myeloma cells were plated in 10-cm plates in RPMI1640 growth media containing LEN (2 μM) or DMSO vehicle control in triplicates. Every 2 days, cells were detached by gentle scraping and half of the total cells were replated in fresh RPMI growth media containing the respective agents (LEN or DMSO) for continuous culturing. At day 2, day 4 and day 8, cells were counted using a ViaCell cell viability counter (Beckman Coulter).

### Results

Fifty years ago, thalidomide was used for insomnia and morning sickness but was later banned because of its teratogenicity, manifest as profound limb defects. Thalidomide and the related drugs lenalidomide and pomalidomide (IMiDs) have regained interest, however, as immunomodulators and antineoplastics, especially for multiple myeloma and other B cell malignancies (1-3). Nonetheless, the biochemical mechanisms underlying their teratogenic and therapeutic activities, and whether they are linked, are unknown.

In this regard, thalidomide was recently shown to bind to cereblon, which is the substrate-recognition component of a cullin-dependent ubiquitin ligase, and to inhibit its autoubiquitination activity (4). Treatment of zebrafish with cereblon morpholinos or thalidomide caused fin defects (4), suggesting that IMiDs act by stabilizing cereblon substrates. However, myeloma cells rendered IMiDs-resistant have frequently down-regulated cereblon (5-8). Conversely, high cereblon concentrations in myeloma cells are associated with increased responsiveness to IMiDs (9, 10). Collectively, these observations suggest that IMiDs are not simply cereblon antagonists but, instead, alter the substrate specificity of cereblon to include proteins important in myeloma.

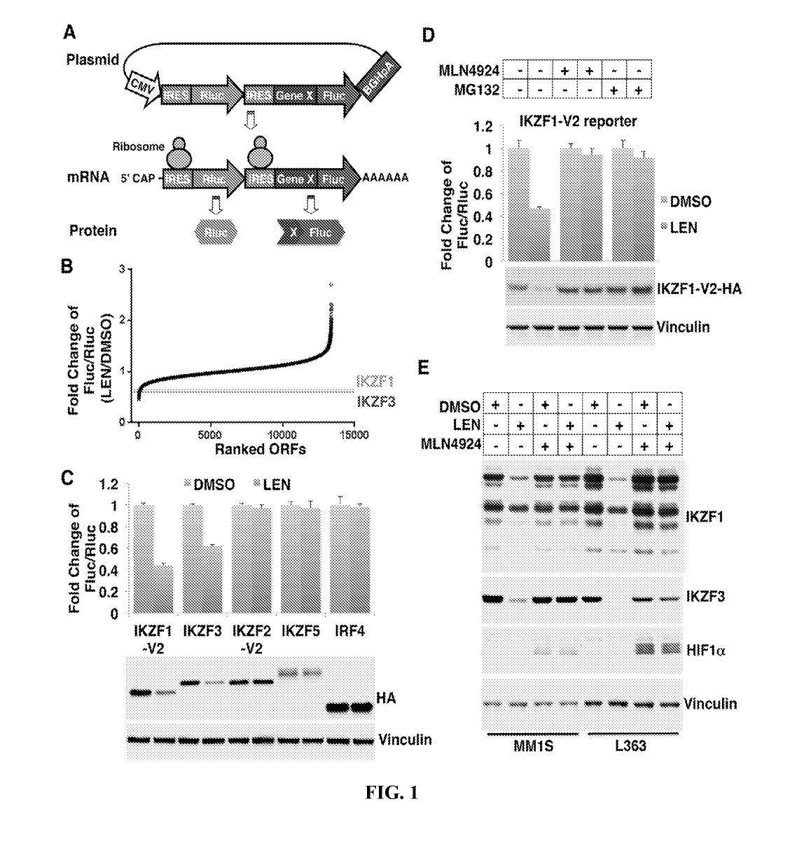

To look for such proteins, a plasmid library encoding 15,483 open reading frames (ORFs) fused to firefly luciferase (Fluc) was made, knowing that the stabilities of such fusions are usually influenced by the ubiquitin ligase(s) for the corresponding unfused ORF (11-13). A renilla luciferase (Rluc) reporter was inserted into each ORF-luciferase cDNA for normalization purposes and placed both reporters under internal ribosome entry site (IRES) control (FIG. 1A).

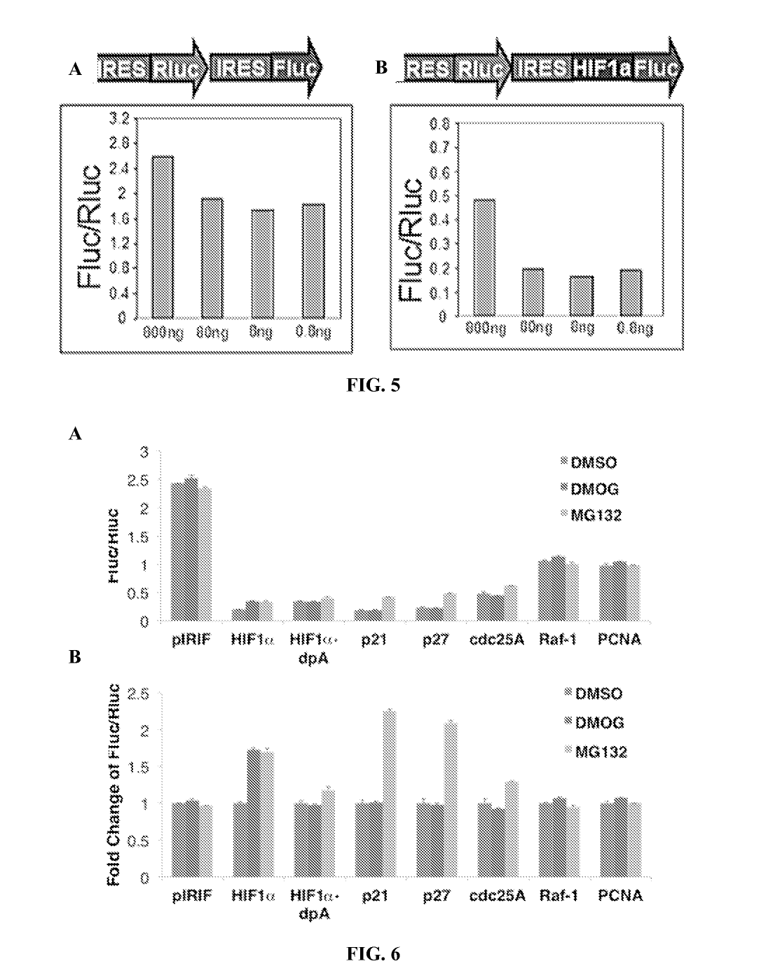

In pilot experiments 293FT embryonic kidney cells grown in multiwell plates were transfected with the ORF-luciferase library (one ORF per well) and treated with the proteasome inhibitor MG132, the hydroxylase inhibitor dimethyloxalylglycine (DMOG), or vehicle. Fluc/Rluc values measured 36 to 48 hours later were stable over a wide range of input plasmid concentrations (FIG. 5). As expected, MG132 stabilized many proteasomal substrates and DMOG stabilized HIF1α, which is rapidly degraded when prolyl hydroxylated (FIG. 6).

Next, this approach was used to identify changes in protein stability in 293FT cells treated with lenalidomide (FIG. 1A). A total of 2113 ORF-luciferase fusions produced luciferase signals that were undetectable or highly variable (>50% SD), leaving 13,370 for analysis. As expected, most ORFs were unaffected by lenalidomide (FIG. 1B). The 107 ORFs that were >3 SDs from the mean (46 ORFs plus 61 ORFs displaying decreased or increased Fluc/Rluc ratios after lenalidomide treatment, respectively) were retested in secondary assays. One down-regulated ORF (IKZF3) and one up-regulated ORF (C11orf65) retested positively.

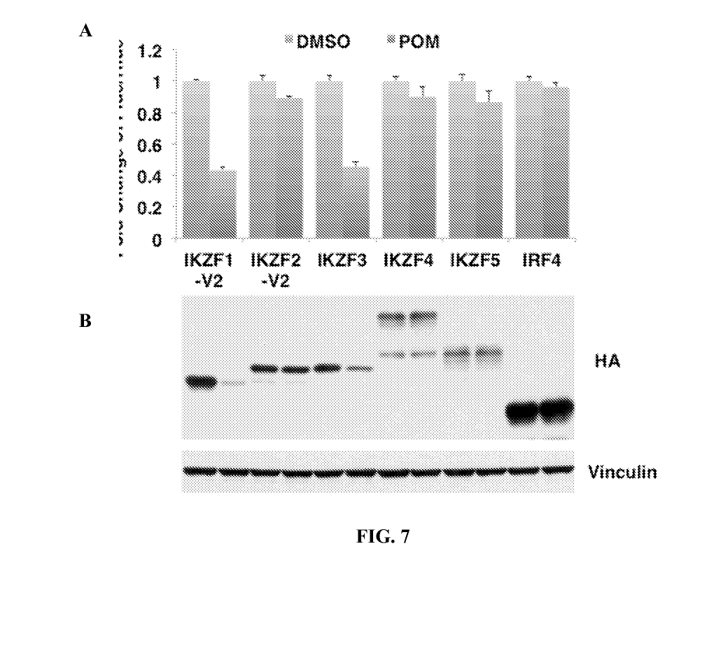

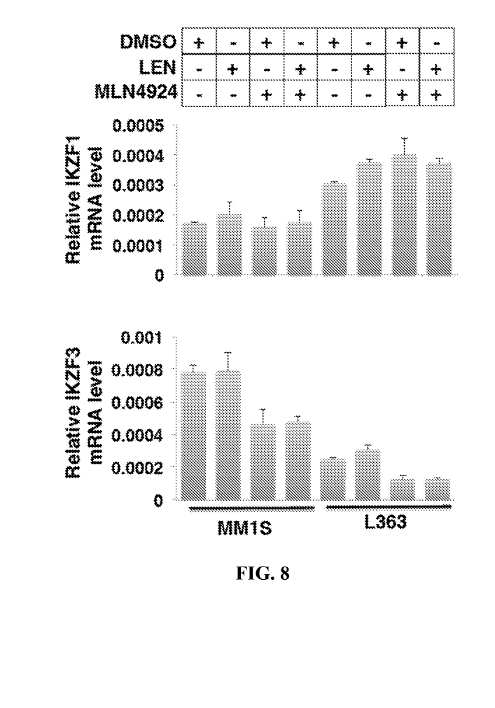

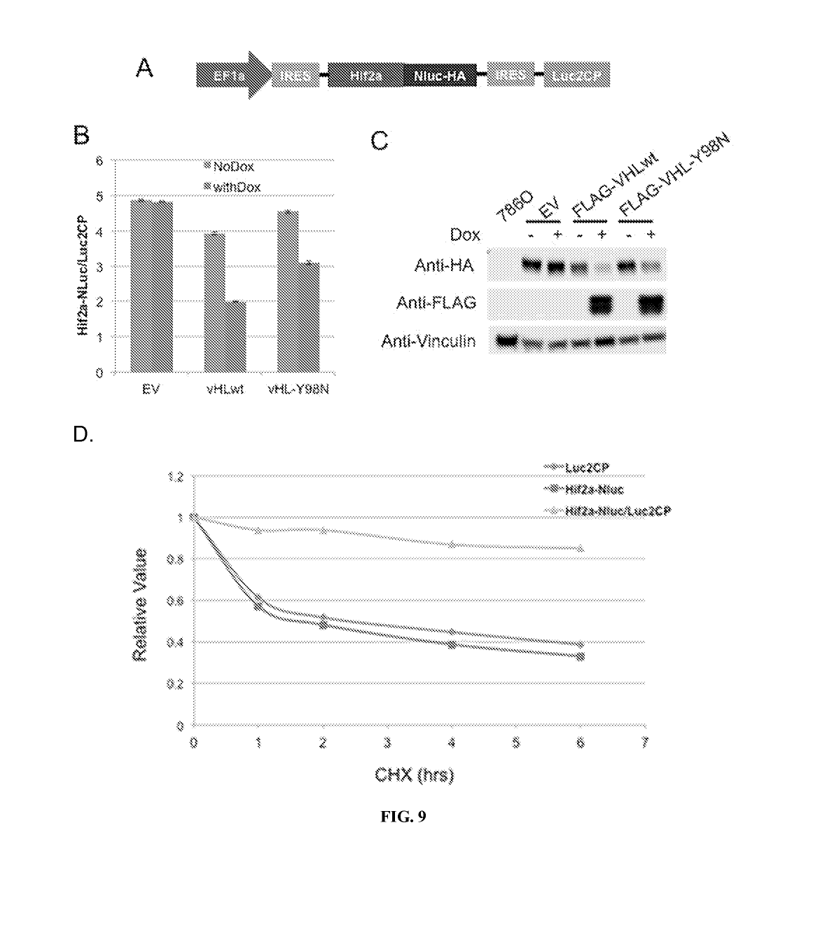

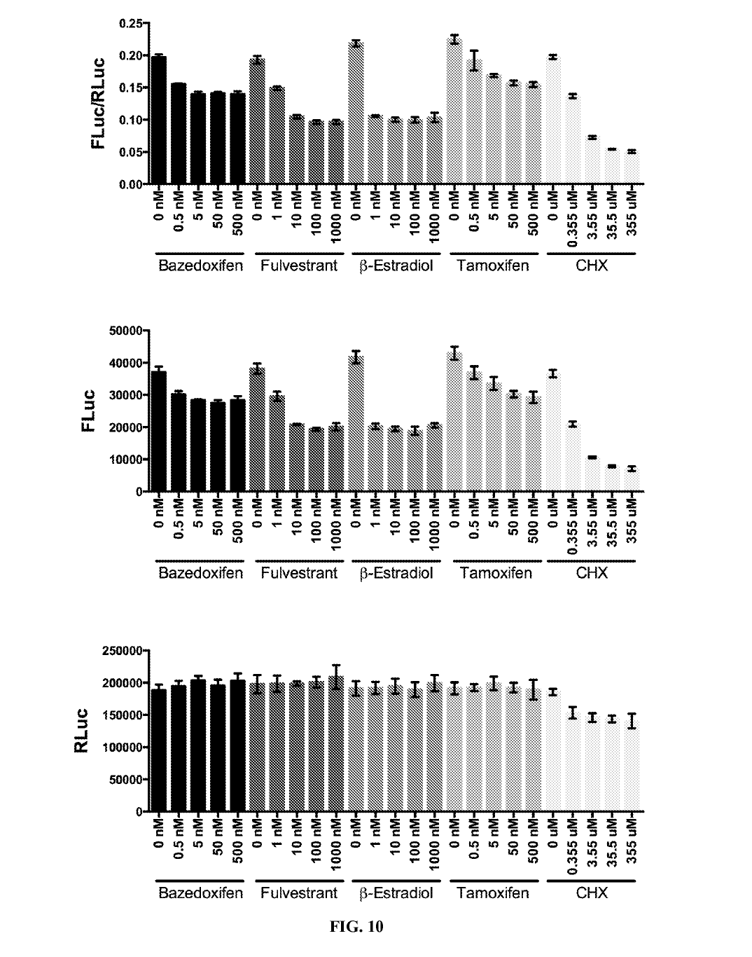

C11orf65 was unaffected by lenalidomide when fused to a hemagglutinin (HA) epitope tag instead of Fluc and so was not studied further. By contrast, lenalidomide down-regulated IKZF3 and its paralog IKZF1, which had fallen just outside the 3-SD cut-off in the primary screen, fused to either Fluc or HA (FIGS. 1B and 1C). These effects were specific because lenalidomide did not affect exogenous IKZF2, IKZF4, IKZF5, or the B cell transcription factor IRF4 (FIG. 1C). Similar results were obtained with two common splice variants (V1 and V2) of IKZF1 and IKZF2 (FIG. 1C) and with pomalidomide (FIG. 7). Down-regulation of exogenous IKZF1 was blocked by MG132 and by MLN4924, which inhibits cullin-dependent ubiquitin ligases (FIG. 1D) (14, 15). Consistent with these findings, lenalidomide down-regulated endogenous IKZF1 in U937 leukemia cells, which do not express IKZF3, and both IKZF1 and IKZF3 in MM1S and L363 myeloma cells (FIG. 1E) unless the cells were pretreated with MG132 or MLN4924 (FIG. 1E). Multiple IKZF1 bands were detected by immunoblot analysis, presumably due to alternative splicing. Lenalidomide did not alter IKZF1 and IKZF3 mRNA levels, consistent with it acting posttranscriptionally (FIG. 8).

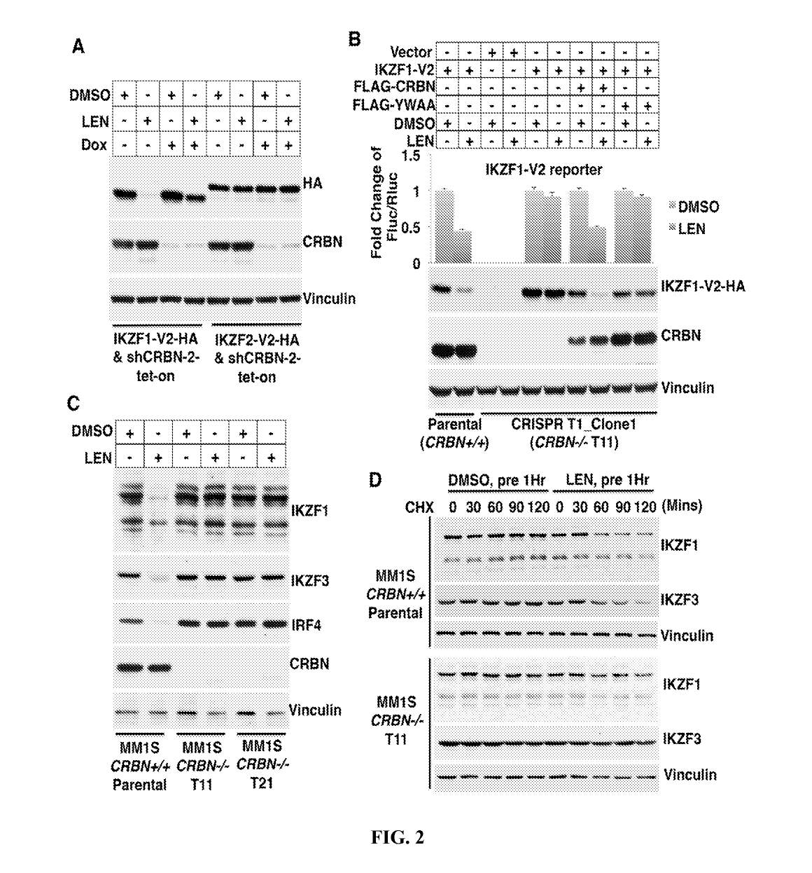

Down-regulation of cereblon in 293FT cells with a doxycycline-inducible short hairpin RNA (shRNA) prevented the destabilization of exogenous, HA-tagged, IKZF1, by lenalidomide (FIG. 2A). Similarly, cereblon shRNA blocked the down-regulation of endogenous IKZF1 by lenalidomide in U937 leukemia cells (U937) and myeloma cells (L363 and KMS34). Clustered regularly interspaced short palindromic repeats (CRISPR)-based gene editing (16, 17) were also used to generate CRBN−/− 293FT cells, which were then transfected to produce IKZF1 fused to Fluc (FIG. 2B, top panel) or HA (FIG. 2B, bottom panel). Exogenous IKZF1 was not down-regulated in CRBN−/− 293FT cells (FIG. 2B). This defect was rescued by wild-type cereblon, but not a lenalidomide-resistant cereblon mutant (YWAA) (4) (FIG. 2B). Similar results were obtained with a second CRBN−/− 293FT subclone, but not in subclones with detectable amounts of cereblon. Moreover, endogenous IKZF1 and IKZF3 were not degraded by lenalidomide in two independent CRBN−/− MM1S myeloma cell lines generated with CRISPR (FIGS. 2C and 2D).

Lenalidomide enhanced the binding of IZKF1 and IKZF3, but not IKZF2 and IKZF5, to the cereblon ubiquitin ligase in cotransfection experiments using MG132-treated 293FT cells. Next, FLAG-tagged IKZF1 and IKZF2 were immunoprecipitated from CRBN−/− cells that were or were not treated with lenalidomide. The immobilized immunoprecipitates were then used to capture endogenous cereblon from CRBN+/+ cells (FIG. 3A) or exogenous cereblon from CRBN−/− cells transfected to produce wild-type cereblon or the YWAA variant (FIG. 3B). In both cases, the cells producing cereblon were or were not treated with lenalidomide before lysis. Wild-type cereblon, but not the YWAA variant, bound specifically to IKZF1 provided that it was exposed to lenalidomide, consistent with lenalidomide binding directly to cereblon rather than to IKZF1 (FIGS. 3A and 3B). Lenalidomide also promoted the binding of cereblon to exogenous IKZF1 and to endogenous IKZF1 and IKZF3 when added directly to binding assays performed with cell extracts. Moreover, wild-type cereblon, but not the YWAA variant, promoted the ubiquitylation of IKZF1 in vivo (FIG. 3C) and in vitro (FIG. 3D) after exposure to lenalidomide.

A series of IKZF1/2 chimeras were analyzed and determined that the region of IKZF1 corresponding to residues 108 to 197 of IKZF1(V2) mediated lenalidomide-dependent binding to cereblon. Within this region, there are only seven amino acid differences between IKZF1 and IKZF2. Changing IKZF1 residue Q146 (or IKZF3 Q147) to the corresponding residue in IKZF2 (histidine) abrogated cereblon binding and lenalidomide-induced degradation. Conversely, the reciprocal change in IKZF2 rendered it partially sensitive to lenalidomide.

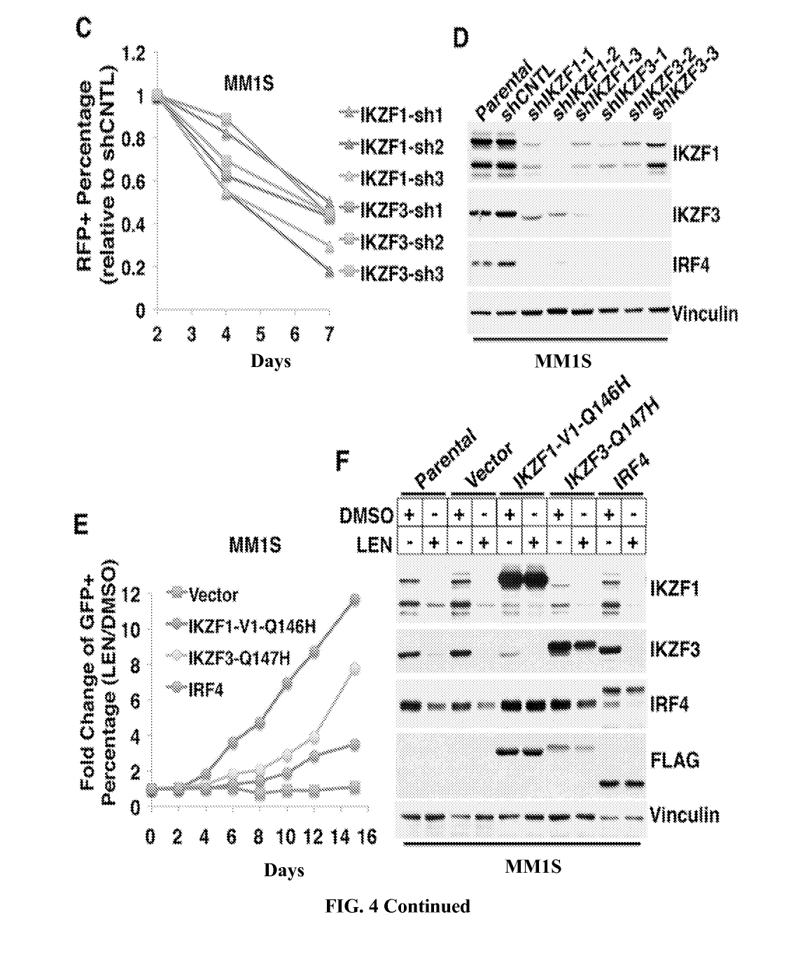

Next, six myeloma cell lines were tested for their sensitivity to lenalidomide in vitro (FIGS. 4A and 4B). Previous studies showed that lenalidomide, at least indirectly, down-regulates IRF4 and linked this to its antimyeloma activity (5-8). MM1S, KMS34, and L363 cells were sensitive to lenalidomide in vitro whereas KMS11, RPMI8226, and OCImy5 cells were relatively resistant (FIG. 4B). In the three sensitive lines, IKZF1 and IKZF3 were down-regulated by lenalidomide (FIG. 4A). In two of these lines (MM1S and KMS34), loss of IKZF1 and IKZF3 was followed by a decrease in IRF4, consistent with IRF4 acting downstream of IKZF1 and/or IKZF3 in these cells (FIG. 4A). Decreased IRF4 mRNA and decreased binding of IKZF1 were confirmed to the IRF4 locus by chromatin immunoprecipitation (ChIP) in MM1S cells treated with lenalidomide. The third sensitive cell line, L363, expressed high basal amounts of IRF4 that were unaffected by lenalidomide, providing evidence that the antiproliferative effects of this drug involves at least one target other than IRF4 (FIG. 4A).

Two of the resistant lines had relatively high basal amounts of IKZF1 (OCImy5) or IKZF3 (KMS11) and corresponding low amounts of cereblon compared to the sensitive lines, and down-regulation of IKZF1 and IKZF3 by lenalidomide was attenuated in the third (RPMI8226) (FIG. 4A). IRF4 was not down-regulated by lenalidomide in the three resistant lines.

Next, competition experiments were performed with cells in which IKZF1 or IKZF3 was suppressed with shRNAs or enhanced through expression of lenalidomide-resistant versions of IKZF1 or IKZF3. Down-regulation of either IKZF1 or IKZF3 in the lenalidomide-sensitive cell lines MM1S and KMS34 markedly decreased cellular fitness compared to cells expressing a control shRNA and was associated with down-regulation of IRF4 (FIGS. 4C and 4D). Notably, down-regulation of either IKZF protein led to loss of the other. Conversely, expression of the stabilized versions of IKZF1(Q146H) or IKZF3(Q147H) conferred lenalidomide resistance to MM1S cells (FIGS. 4E and 4F) and KMS34 cells. Ectopic expression of a T cell-specific Ikaros family member, IKZF2, which is naturally lenalidomide resistant (FIG. 1C), had similar effects. The effects of expressing IRF4 itself were much less pronounced, again suggesting that IKZF1 and IKZF3 have additional targets that are relevant for lenalidomide's antimyeloma activity (FIG. 4E). It remains to be seen whether lenalidomide-resistance conferred by IKZF family members is due primarily to transcriptional activation of their target genes or to noncanonical functions.

The findings link lenalidomide's antimyeloma activity to down-regulation of IKZF1 and IKZF3, two transcription factors that play critical roles in B cell development and are highly expressed in B cell malignancies, including myeloma (18-21). There are many other examples of cancers that become addicted to transcription factors that specify cell lineage (22, 23). Although IKZF1 is a tumor suppressor in some other B cell malignancies (24), there is precedence for the same gene acting as either a tumor suppressor or an oncogene in different contexts.

Ikaros family members can serve as transcriptional activators or repressors in different settings. For example, IKZF1 and IKZF3 repress interleukin-2 (IL-2) expression in T cells, thus explaining how IMiDs induce IL-2 production in vivo (19, 25, 26).

The proteasomal inhibitor bortezomib has antimyeloma activity, alone and in combination with lenalidomide, although the pertinent proteasomal substrates are debated (27, 28). This creates a paradox because proteasomal blockade prevents the destruction of IKZF1 and IKZF3 by lenalidomide. Proteasomal blockade by bortezomib is incomplete with therapeutic dosing, however, which might allow sufficient clearance of IKZF1 and IKZF3 while retaining bortezomib's other salutary effects. It is also possible that these two proteins, once polyubiquitylated, are inactive or dominant-negatives.

Earlier work suggested that thalidomide's teratogenic effects reflected cereblon inactivation, whereas these findings indicate that the therapeutic effects of the IMiDs reflect a cereblon gain of function. Notably, cereblon might have additional substrates that were not in the fusion library, could not be recognized as luciferase fusions, or require accessory proteins or signals absent in 293FT cells. Regardless, the findings create a path to uncouple the therapeutic and teratogenic activities of the IMiDs. It is not yet clear whether lenalidomide's effect on cereblon is hypermorphic or neomorphic. Precedence for the latter is provided by rapamycin, which converts FKBP12 into a TORC1 kinase inhibitor and cyclosporine, which converts cyclophylin into a calcineurin antagonist (29). Perhaps oncoproteins currently deemed undruggable, such as c-Myc or β-catenin, could be destroyed by drugs that, like lenalidomide, repurpose ubiquitin ligases.

### REFERENCES

1. R. Martiniani, V. Di Loreto, C. Di Sano, A. Lombardo, A. M. Liberati, Biological activity of lenalidomide and its underlying therapeutic effects in multiple myeloma. Adv Hematol 2012, 842945 (2012).
2. E. Terpos, N. Kanellias, D. Christoulas, E. Kastritis, M. A. Dimopoulos, Pomalidomide: a novel drug to treat relapsed and refractory multiple myeloma. OncoTargets and therapy 6, 531 (2013).
3. Y. X. Zhu, K. M. Kortuem, A. K. Stewart, Molecular mechanism of action of immune-modulatory drugs thalidomide, lenalidomide and pomalidomide in multiple myeloma. Leukemia & lymphoma 54, 683 (April, 2013).
4. T. Ito et al., Identification of a primary target of thalidomide teratogenicity. Science 327, 1345 (Mar. 12, 2010).
5. A. Lopez-Girona et al., Cereblon is a direct protein target for immunomodulatory and antiproliferative activities of lenalidomide and pomalidomide. Leukemia 26, 2326 (November, 2012).
6. L. H. Zhang et al., Lenalidomide efficacy in activated B-cell-like subtype diffuse large B-cell lymphoma is dependent upon IRF4 and cereblon expression. Br J Haematol 160, 487 (February, 2013).
7. Y. Yang et al., Exploiting synthetic lethality for the therapy of ABC diffuse large B cell lymphoma. Cancer Cell 21, 723 (Jun. 12, 2012).
8. Y. X. Zhu et al., Cereblon expression is required for the antimyeloma activity of lenalidomide and pomalidomide. Blood 118, 4771 (Nov. 3, 2011).
9. A. Broyl et al., High cereblon expression is associated with better survival in patients with newly diagnosed multiple myeloma treated with thalidomide maintenance. Blood 121, 624 (Jan. 24, 2013).
10. D. Heintel et al., High expression of cereblon (CRBN) is associated with improved clinical response in patients with multiple myeloma treated with lenalidomide and dexamethasone. Br J Haematol 161, 695 (Jun., 2013).
11. G. J. Zhang et al., Bioluminescent imaging of Cdk2 inhibition in vivo. Nat Med 10, 643 (Jun., 2004).
12. M. Safran et al., Mouse model for noninvasive imaging of HIF prolyl hydroxylase activity: assessment of an oral agent that stimulates erythropoietin production. Proc Natl Acad Sci USA 103, 105 (Jan. 3, 2006).
13. H. C. Yen, Q. Xu, D. M. Chou, Z. Zhao, S. J. Elledge, Global protein stability profiling in mammalian cells. Science 322, 918 (Nov. 7, 2008).
14. T. A. Soucy et al., An inhibitor of NEDD8-activating enzyme as a new approach to treat cancer. Nature 458, 732 (Apr. 9, 2009).
15. M. Ohh et al., An intact NEDD8 pathway is required for Cullin-dependent ubiquitylation in mammalian cells. EMBO reports 3, 177 (Feb., 2002).
16. L. Cong et al., Multiplex genome engineering using CRISPR/Cas systems. Science 339, 819 (Feb. 15, 2013).
17. P. Mali et al., RNA-guided human genome engineering via Cas9. Science 339, 823 (Feb. 15, 2013).
18. M. Merkenschlager, Ikaros in immune receptor signaling, lymphocyte differentiation, and function. FEBS Lett 584, 4910 (Dec. 15, 2010).
19. E. C. Thompson et al., Ikaros DNA-binding proteins as integral components of B cell developmental-stage-specific regulatory circuits. Immunity 26, 335 (Mar., 2007).
20. I. Ferreiros-Vidal et al., Genome-wide identification of Ikaros targets elucidates its contribution to mouse B-cell lineage specification and pre-B-cell differentiation. Blood 121, 1769 (Mar. 7, 2013).
21. S. Monticelli, F. Sallusto, Negative regulators take center stage. Nature immunology 13, 719 (August, 2012).
22. L. A. Garraway, W. R. Sellers, Lineage dependency and lineage-survival oncogenes in human cancer. Nat Rev Cancer 6, 593 (August, 2006).
23. S. P. Shah et al., Mutation of FOXL2 in granulosa-cell tumors of the ovary. N Engl J Med 360, 2719 (Jun. 25, 2009).
24. P. Kastner et al., Function of Ikaros as a tumor suppressor in B cell acute lymphoblastic leukemia. American journal of blood research 3, 1 (2013).
25. F. J. Quintana et al., Aiolos promotes TH17 differentiation by directly silencing I12 expression. Nature immunology 13, 770 (Aug., 2012).
26. N. Avitahl et al., Ikaros sets thresholds for T cell activation and regulates chromosome propagation. Immunity 10, 333 (Mar., 1999).
27. J. Laubach, P. Richardson, K. Anderson, Multiple myeloma. Annu Rev Med 62, 249 (2011).
28. P. G. Richardson et al., Multicenter, phase I, dose-escalation trial of lenalidomide plus bortezomib for relapsed and relapsed/refractory multiple myeloma. J Clin Oncol 27, 5713 (Dec. 1, 2009).
29. G. R. Crabtree, S. L. Schreiber, Three-part inventions: intracellular signaling and induced proximity. Trends Biochem Sci 21, 418 (Nov., 1996).
30. M. R. Campanero, E. K. Flemington, Regulation of E2F through ubiquitin-proteasome-dependent degradation: stabilization by the pRB tumor suppressor protein. Proc Natl Acad Sci USA 94, 2221 (Mar. 18, 1997).
31. D. Wiederschain et al., Single-vector inducible lentiviral RNAi system for oncology target validation. Cell Cycle 8, 498 (Feb. 1, 2009).
32. C. J. Ott et al., BET bromodomain inhibition targets both c-Myc and IL7R in high-risk acute lymphoblastic leukemia. Blood 120, 2843 (Oct. 4, 2012).
33. K. Kondo, J. Klco, E. Nakamura, M. Lechpammer, W. G. Kaelin, Inhibition of HIF is necessary for tumor suppression by the von Hippel-Lindau protein. Cancer Cell 1, 237 (Apr., 2002).
34. J. Loven et al., Selective inhibition of tumor oncogenes by disruption of super-enhancers. Cell 153, 320 (Apr. 11, 2013).

1. A DNA plasmid comprising in operable linkage:
(a) a promoter;
(b) a first internal ribosomal entry site (IRES);
(c) a nucleotide sequence encoding a first reporter protein;
(d) a second IRES; and
(e) a nucleotide sequence encoding a second reporter protein,
wherein an open reading frame (ORF) is fused to the nucleotide sequence encoding a first reporter protein or to the nucleotide sequence encoding a second reporter protein.

2. The DNA plasmid of claim 1, wherein said first and second reporter proteins have distinguishable detectable reporter signals.

3. The DNA plasmid of claim 2, wherein said first and second reporter proteins are:
(i) enzyme proteins having distinguishable signals generated from their products; or
(ii) bioluminescent proteins having distinguishable bioluminescence signals; or
(iii) fluorescent proteins having distinguishable fluorescence signals.

4-5. (canceled)

6. The DNA plasmid of claim 2, wherein the first and second reporter proteins are:
(i) bioluminescent proteins selected from the group consisting of renilla luciferase (Rluc) and firefly luciferase (FLuc); or
(ii) fluorescent proteins selected from the group consisting of green fluorescence protein and red fluorescence protein.

7. (canceled)

8. The DNA plasmid of claim 1, wherein the promoter is a eukaryotic promoter or a synthetic promoter.

9. The DNA plasmid of claim 8, wherein the promoter comprises cytomegalovirus (CMV) promoter.

10. The DNA plasmid of claim 1, wherein said ORF is derived from an ORFeome of an organism.

11. The DNA plasmid of claim 1, wherein said ORF encodes an oncoprotein.

12. The DNA plasmid of claim 11, wherein said oncoprotein is selected from the group consisting of MYC, Ikaros family zinc finger protein 1 (IKZF1), Ikaros family zinc finger protein 3 (IKZF3), Interferon regulatory factor 4 (IRF4), mutant p53, N-Ras, c-Fos, and c-Jun.

13. An isolated transformed host cell comprising the DNA plasmid of claim 1.

14. The isolated transformed host cell of claim 13, wherein the host cell is a bacterial cell, a yeast cell, a plant cell, an insect cell, or a mammalian cell.

15. A DNA plasmid library comprising a plurality of plasmids, wherein each said plasmid comprises in operable linkage:
(a) a promoter;
(b) a first internal ribosomal entry site (IRES);
(c) a nucleotide sequence encoding a first reporter protein;
(d) a second IRES; and
(e) a nucleotide sequence encoding a second reporter protein,
wherein an open reading frame (ORF) is fused to the nucleotide sequence encoding a first reporter protein or to the nucleotide sequence encoding a second reporter protein.

16. The DNA plasmid library of claim 15, wherein said ORF of each plasmid is different.

17. The DNA plasmid library of claim 15, wherein said ORF of each plasmid is derived from an ORFeome of an organism, optionally wherein said organism is a human.

18-24. (canceled)

25. The DNA plasmid library of claim 15, wherein the promoter is a eukaryotic promoter or a synthetic promoter.

26. The DNA plasmid library of claim 25, wherein the promoter comprises cytomegalovirus (CMV) promoter.

27. A method for identifying proteins whose levels are modulated by a compound of interest, the method comprising:
(i) contacting host cells transformed with the DNA plasmid library of claim 15 with a compound of interest;
(ii) determining ratios of fused reporter protein signal to unfused reporter protein signal in presence and absence of the compound; and
(iii) identifying open reading frames that have increased levels when the ratio of fused reporter protein signal to unfused reporter protein signal in the presence of the compound is increased as compared to the ratio of fused reporter protein signal to unfused reporter protein signal in the absence of the compound and identifying open reading frames that have decreased levels when the ratio of fused reporter protein signal to unfused reporter protein signal in the presence of the compound is decreased as compared to the ratio of fused reporter protein signal to unfused reporter protein signal in the absence of the compound.

28. The method of claim 27, wherein contacting host cells transformed with the DNA plasmid library with a compound of interest comprises growing the transformed host cells in the presence of the compound for an appropriate time and/or wherein the compound of interest is an IMiD.

29. (canceled)

30. A method for monitoring treatment of a subject with an IMiD compound, the method comprising:
determining in a sample of a subject treated with an IMiD compound a level of IKZF1 and/or IKZF3; and
identifying the subject as responding to the treatment when the level of IKZF1 and/or IKZF3 is decreased as compared to a reference level.

31. The method of claim 30, wherein the reference level is the level of IKZF1 and/or IKZF3 in a control subject that has not been treated with the IMiD compound.
